(19) United States
(12) Patent Application Publication
(10) Pub. No.: US 2026/0288856 A1
(43) Pub. Date: Sep. 24, 2026

(54) SYSTEM

(71) Applicant: SoftBank Group Corp., Tokyo (JP)

(72) Inventor: Kohei HAGIHARA, Tokyo (JP)

(73) Assignee: SoftBank Group Corp., Tokyo (JP)

(21) Appl. No.: 19/564,474

(22) Filed: Mar. 12, 2026

(30) Foreign Application Priority Data
Mar. 19, 2025 (JP) 2025-044941

(51) Int. Cl. G06F 16/435; G06F 16/438; G06F 16/45

(52) U.S. Cl. CPC G06F 16/435; G06F 16/438; G06F 16/45

(57) ABSTRACT

A system includes a processor that is configured to: collect daily life data of a user through a sensor device and an application, and analyze the collected data to manage an amount of exercise of the user and meal content of the user; analyze voice data and text data of the user to recognize an emotion of the user and adjust content of advice and encouragement based on a recognition result, and generate a prompt for providing the advice and the encouragement to the user by using a generative artificial intelligence model; and generate, by using the prompt, a coach that imitates a person or a character preferred by the user.

### CROSS-REFERENCE TO RELATED APPLICATION

This application is based on and claims priority under 35 USC 119 from Japanese Patent Application No. 2025-044941 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional health management systems mainly focus on recording and displaying numerical data such as step counts, calories, and simple meal logs, and therefore have limited ability to provide continuously effective motivation or personalized guidance that reflects the user's emotional state and preferences. Known systems typically lack integrated processing of heterogeneous data sources, such as sensor-derived activity data, image-based meal information, delivery service histories, and electronic payment records, and thus cannot accurately grasp the user's actual lifestyle and dietary habits. Furthermore, existing systems often provide uniform advice and notifications that do not take into account an emotional condition of the user, a communication style preferred by the user, or a favorite person or character of the user, which results in low engagement and poor continuity in health behavior change. There is a need for a system that can comprehensively collect and analyze daily life data, recognize the user's emotion from voice data and text data, and dynamically tailor advice and encouragement, while also presenting the guidance through a coach that imitates a person or character preferred by the user so as to enhance motivation and adherence over the long term.

### SUMMARY

To solve the above-described problems, a system according to one aspect of the present invention comprises a processor, wherein the processor is configured to collect daily life data of a user through a sensor device and an application, and analyze the collected data to manage an amount of exercise of the user and meal content of the user. The processor is further configured to analyze voice data and text data of the user to recognize an emotion of the user and adjust content of advice and encouragement based on a recognition result. In addition, the processor is configured to generate a prompt for providing the advice and the encouragement to the user by using a generative artificial intelligence model, and generate, by using the prompt, a coach that imitates a person or a character preferred by the user. In some embodiments, the processor acquires, in real time, data representing the amount of exercise of the user by cooperation with a sensor of a mobile information terminal, and manages the amount of exercise of the user. In some embodiments, the processor analyzes image data representing a meal of the user by using image recognition technology to obtain the meal content of the user, acquires a history of a delivery service and purchase information of an electronic payment service from a database, and calculates and manages the meal content of the user based on the image data, the history, and the purchase information. By these means, the system can provide highly personalized, emotionally adaptive advice and encouragement through a user-preferred coach, thereby improving user engagement and continuity of health behavior.

The term “system” refers to an electronic apparatus including at least one processor and, optionally, one or more memories, communication interfaces, and input/output devices, configured to execute the processing steps described in the claims.

The term “processor” refers to any hardware capable of executing instructions, including but not limited to a central processing unit (CPU), a graphics processing unit (GPU), a digital signal processor (DSP), or a combination thereof, whether implemented as a single device or a distributed set of devices.

The term “daily life data” refers to data representing activities, behaviors, and conditions of a user in everyday life, including but not limited to physical activity data, movement data, sleep data, location data, and meal-related data.

The term “sensor device” refers to any device having one or more sensors capable of measuring physical quantities associated with the user or the environment, including but not limited to accelerometers, gyroscopes, heart rate sensors, GPS modules, and similar components.

The term “application” refers to software executed on a terminal or other computing device for collecting, transmitting, displaying, or managing data related to the user, including mobile applications, desktop applications, and web applications.

The term “amount of exercise” refers to a quantitative measure of physical activity of the user, including but not limited to step count, distance traveled, active minutes, energy expenditure, or any combination thereof.

The term “meal content” refers to information representing the type, quantity, and nutritional characteristics of food and beverages consumed by the user, including but not limited to calories, macronutrient composition, and estimated servings of specific food groups.

The term “voice data” refers to audio data including speech uttered by the user, captured by a microphone or other audio input device, and used for analysis of content or emotion.

The term “text data” refers to textual information generated or input by the user, including but not limited to chat messages, comments, diary entries, and responses to questionnaires.

The term “emotion” refers to an emotional state or affective condition of the user, such as happiness, sadness, anger, stress, or calmness, inferred from analysis of voice data and/or text data.

The term “advice” refers to information presented to the user that suggests actions, changes in behavior, or improvements in lifestyle, including but not limited to recommendations regarding exercise, diet, rest, and daily habits.

The term “encouragement” refers to information presented to the user for the purpose of motivating, praising, or supporting the user, including but not limited to congratulatory messages, motivational comments, and reinforcements related to achievements.

The term “generative artificial intelligence model” refers to a machine learning model configured to generate data, such as text, images, or audio, based on input data or prompts, including but not limited to large language models and other generative models.

The term “prompt” refers to input information provided to the generative artificial intelligence model, including instructions, conditions, and contextual data, used to control or guide the content of advice, encouragement, or coach generation.

The term “coach” refers to a virtual entity generated by the system that provides advice and encouragement to the user, and that is represented at least in text, image, or audio form.

The term “person or character preferred by the user” refers to an actual or fictional individual, including but not limited to celebrities, anime characters, game characters, and original characters, selected or specified directly or indirectly by the user as a preferred model for the coach.

The term “mobile information terminal” refers to a portable electronic device capable of communication and data processing, including but not limited to smartphones, tablet computers, and smartwatches.

The term “image data representing a meal” refers to digital image data capturing at least part of food or beverages to be consumed or being consumed by the user.

The term “image recognition technology” refers to a technique for analyzing image data by using algorithms or machine learning models to detect, classify, or estimate objects, food items, or quantities included in the image.

The term “history of a delivery service” refers to record data indicating past use of a food or meal delivery service by the user, including at least order times, item names, quantities, and vendor information.

The term “purchase information of an electronic payment service” refers to transaction data acquired from an electronic payment platform, including at least purchase times, store names, amounts, and item descriptions or categories related to food or meals.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

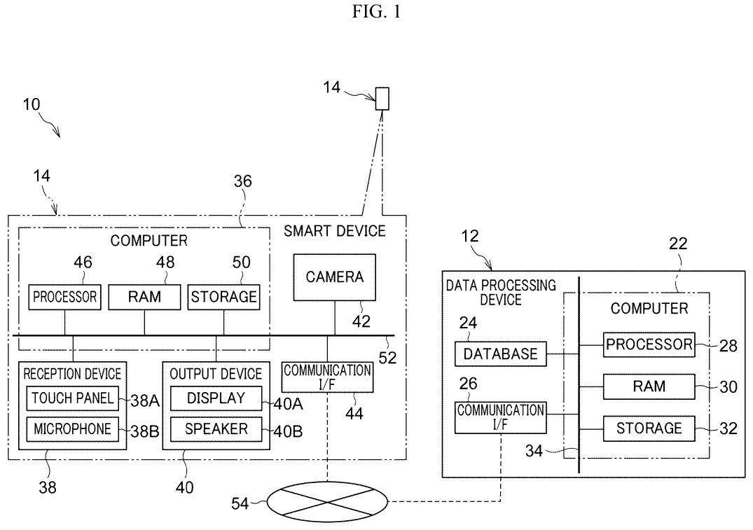

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

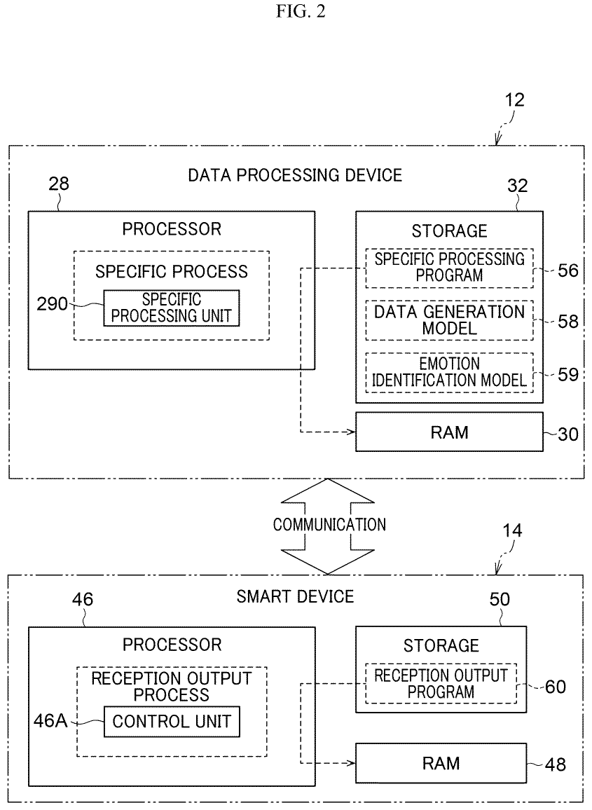

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional health management systems that utilize networked computers and mobile terminals typically treat sensor data collection, dietary logging, and feedback presentation as loosely coupled functions. Activity sensors and location sensors generate raw time-series data at the device level, separate dietary logs are sometimes captured as unstructured images or free-text, and back-end servers often perform only simple aggregation and threshold comparison. As a result, the computing architecture does not fully exploit heterogeneous data sources, and the generated feedback is generic, poorly contextualized, and computationally inefficient to produce at scale.

In particular, existing systems that incorporate generative AI models generally invoke such models in an ad hoc manner, by sending unstructured prompts without a systematic procedure for: (i) converting multi-modal lifestyle data into normalized, machine-readable summaries; (ii) embedding those summaries into prompt sentences in a way that reliably constrains the model's task; and (iii) post-processing AI outputs into stable, structured artifacts that can be persistently managed and re-used. This lack of a coherent processing pipeline leads to redundant computation on the server, difficulty in enforcing safety and style constraints, and inconsistent quality of generated advice.

Furthermore, typical server-side architectures do not natively support a mechanism by which a virtual instructor persona is dynamically synthesized and maintained as a first-class computational object. Persona styling is often hard-coded in application logic or user interface assets and not integrated into the AI prompting workflow. Consequently, servers are unable to programmatically adapt the tone, structure, and granularity of the generated advice to individual user preferences without duplicating substantial application code or manually tuning prompts for each case.

From a computer technology standpoint, there is a need for an improved server-centric processing framework that: (i) systematically acquires and consolidates heterogeneous lifestyle-related time-series data into unified structured data; (ii) programmatically constructs and manages task-specific prompt sentences based on computed lifestyle indices and health state indices; (iii) orchestrates multiple interactions with a generative AI model, including evaluation, advice generation, and persona-specific re-styling, as well-defined server-side operations; and (iv) delivers finalized advice to user terminals in a form that is both machine-manageable and user-personalized. Without such a framework, server resources are underutilized, the reproducibility and controllability of AI-generated content are limited, and the overall performance and scalability of health-advice generation services remain insufficient.

The specific processing by the specific processing unit 290 of the data processing device 12 in Example 1 is realized by the following means.

The present invention provides a server comprising a processor and a memory storing instructions that, when executed by the processor, cause the processor to acquire time-series data relating to a user's daily life from a plurality of information acquisition devices via a communication interface, integrate the time-series data per user and per period into structured data including body activity data, location data, intake data, and transaction data, and store the structured data in a storage device; to analyze the structured data to compute aggregated body activity values, movement amounts, intake contents, and estimated nutrition values, and to classify the intake contents by applying an image analysis algorithm to intake images and by performing a collation process with transaction information so as to generate lifestyle indices and health state indices for the user; to construct one or more prompt sentences that specify task content and output format by embedding summary data including the lifestyle indices and the health state indices into a predefined prompt template, thereby generating a model input sentence; to transmit the model input sentence to a generative AI model via an AI interface, receive a natural-language evaluation result and advice content from the generative AI model, and parse the natural-language evaluation result and the advice content to extract health evaluation information and lifestyle improvement proposal information as structured advice data; to generate additional prompt sentences including style information indicating attribute information of a virtual instructor persona, apply the additional prompt sentences and the structured advice data to the generative AI model to produce a user-personalized advice text that imitates a human image or character image preferred by the user; and to transmit the advice text to an information display terminal via the communication interface so that the information display terminal presents the advice text to the user. This enables an integrated, server-controlled computational pipeline that transforms heterogeneous, multi-modal lifestyle data into standardized, prompt-ready summaries, systematically orchestrates interactions with a generative AI model for evaluation and persona-specific advice generation, and delivers consistent, personalized guidance while improving the efficiency, reproducibility, and scalability of computer-based health management and feedback systems.

The term “time-series data” refers to data elements that are associated with timestamps and are collected in chronological order over a period of time, such that temporal changes in a user's state or behavior can be analyzed.

The term “user's daily life” refers to day-to-day activities and behaviors of a user, including but not limited to physical activity, movement, eating and drinking, and commercial transactions that occur in ordinary living environments.

The term “information acquisition device” refers to an electronic device having one or more detection functions that is capable of obtaining data relating to a user's daily life, and includes, for example, portable terminals, wearable devices, and sensor-equipped computing devices.

The term “detection function” refers to a hardware or software function that obtains measurements or observations of physical or digital phenomena, including but not limited to motion sensing, location sensing, image capturing, and transaction logging.

The term “information processing program” refers to software instructions executed by a processor of an information acquisition device or a server to control data collection, formatting, communication, analysis, and storage of user-related information.

The term “structured data” refers to data that is organized in a predefined format, such as records, fields, or key-value pairs, so that individual elements such as body activity data, location data, intake data, and transaction data can be programmatically accessed and processed.

The term “body activity data” refers to data representing physical activity of a user, including but not limited to step counts, movement intensity, active time, and other metrics derived from motion or physiological sensors.

The term “location data” refers to data indicating a physical position or movement of a user or device, including but not limited to latitude, longitude, and derived movement distances.

The term “intake data” refers to data relating to consumption of food or drink by a user, including information describing food items, meal times, quantities, and associated images.

The term “transaction data” refers to data representing commercial activities associated with a user, including but not limited to purchase records, order histories, item descriptions, prices, and transaction timestamps.

The term “aggregated values of body activity” refers to summary metrics computed from raw body activity data over a specified period, such as total steps, total active minutes, or average activity level.

The term “amounts of location movement” refers to quantitative indicators of movement derived from location data, including distances traveled or area coverage during a specified period.

The term “intake contents” refers to the types and characteristics of foods and drinks consumed by a user, including categories of items, portion sizes, and nutritional attributes.

The term “estimated nutrition values” refers to estimates of nutritional components associated with intake contents, including but not limited to calories, macronutrients, and micronutrients.

The term “image analysis algorithm” refers to a computational procedure that processes image data to extract information such as detected objects, categories, or estimated quantities, and includes, for example, image recognition and segmentation techniques.

The term “collation process with transaction information” refers to a processing step in which intake-related image analysis results are associated with transaction data such as purchase or delivery records in order to refine identification and characterization of intake contents.

The term “lifestyle indices” refers to one or more quantitative or qualitative indicators computed from structured data that summarize aspects of a user's lifestyle, including activity patterns, movement patterns, and intake patterns.

The term “health state indices” refers to one or more indicators that characterize a user's health-related condition or tendencies, estimated on the basis of lifestyle indices and other user data, without necessarily providing a medical diagnosis.

The term “summary data” refers to a compact representation of a user's lifestyle indices, health state indices, and related metrics over a specified period, suitable for inclusion in a prompt sentence.

The term “prompt sentence” refers to a text instruction provided as input to a generative AI model, the text instruction specifying a task, constraints, or an output format, and optionally embedding structured or summary data.

The term “model input sentence” refers to a complete textual input to a generative AI model that includes at least one prompt sentence and incorporates summary data or other user-related information required for the model to perform a requested task.

The term “generative artificial intelligence model” refers to a computational model that generates text or other content in response to input prompts by using machine learning techniques such as deep learning, including but not limited to large language models.

The term “natural language generation function” refers to a function of a computational model that outputs text in a human language based on given input data or prompts.

The term “natural language evaluation result” refers to a text output produced by a generative artificial intelligence model that expresses an assessment or analysis of a user's lifestyle indices or health state indices in a human-readable language.

The term “advice contents” refers to text output produced by a generative artificial intelligence model that provides suggestions, recommendations, or guidance for improving or maintaining a user's lifestyle or health-related behavior.

The term “health evaluation information” refers to structured information derived from a natural language evaluation result and indicating the condition or tendencies of a user's health-relevant behavior.

The term “lifestyle improvement proposal information” refers to structured information derived from advice contents and indicating specific changes or actions that a user may take to improve lifestyle-related factors.

The term “structured advice data” refers to advice-related information that has been converted from natural language output into organized fields, categories, or records suitable for storage, retrieval, and further processing by a server.

The term “virtual instructor persona” refers to a conceptual representation of an instructor or coach having certain attributes such as name, tone, personality, or style, which is used to control the way a generative artificial intelligence model phrases or presents advice.

The term “style information” refers to data describing attributes of a virtual instructor persona, including, for example, formality level, friendliness, motivational tone, and preferred linguistic style.

The term “attribute information of a virtual instructor” refers to properties associated with a virtual instructor persona, such as role, personality traits, and communication style, that influence presentation of advice.

The term “advice text” refers to a natural language text produced by or through a generative artificial intelligence model and server processing, where the text provides user-specific guidance or feedback, optionally styled according to a virtual instructor persona.

The term “information display terminal” refers to an electronic device equipped with a display function and a communication function that can receive advice text from a server and present the advice text to a user, including, for example, portable terminals and stationary computing devices.

The term “portable information terminal” refers to a user-carried electronic device having communication and detection functions, such as a handheld device or a wearable device, capable of collecting and transmitting user-related data.

The term “encrypted communication” refers to a communication method in which data is transformed using cryptographic techniques so that the data is unintelligible to unauthorized parties during transmission.

The term “server device” refers to a computing apparatus, which may be implemented by one or more physical or virtual machines, that executes server-side processes including data acquisition, storage, analysis, and generation of advice text.

The term “image analysis function” refers to a software-implemented function executed by a processor that performs image recognition or related processing on image data to infer semantic information such as food categories or portion sizes.

The term “information storage device” refers to a hardware or virtual storage resource that retains digital data such as delivery service histories, electronic payment histories, structured data, or advice logs for subsequent retrieval and processing.

The term “delivery service history” refers to stored information relating to orders placed by a user with a delivery service, including items ordered, times, and associated transaction details.

The term “electronic payment history” refers to stored information relating to payments made by a user through an electronic payment service, including transaction identifiers, items, amounts, and timestamps.

The term “transaction information” refers to data extracted from delivery service histories and electronic payment histories that describes one or more commercial transactions, including purchased items and related attributes.

The term “intake items” refers to specific foods or drinks that a user consumes, identified or inferred from image data, transaction information, or both.

The term “intake amounts” refers to estimated quantities of intake items consumed by a user, such as portion size or volume, determined based on image analysis, transaction information, or derived rules.

The term “nutrition components” refers to nutritional constituents of intake items, including but not limited to caloric content, macronutrients, and micronutrients.

The term “quantitatively manage the intake contents” refers to a process in which intake contents are represented and handled as numeric or categorized values that can be aggregated, compared, or monitored over time by a computing system.

The server implements embodiments of the invention as one or more computer programs executed by one or more processors. The server uses a memory device to store the computer programs, intermediate data, user profiles, lifestyle indices, health state indices, and advice logs. The server communicates with one or more terminals and with a generative AI model through a network interface supporting wired or wireless communication.

The terminal is, for example, a smartphone, a tablet device, or a wearable device such as a smart watch. The terminal includes hardware such as an accelerometer, a gyroscope, a heart-rate sensor, a global positioning system (GPS) receiver, a camera, a wireless communication interface (for example, Wi-Fi, cellular, or Bluetooth), and a display. The terminal executes an application program that collects sensor data, acquires image data, and transmits structured data to the server. In particular embodiments, the terminal runs an application implemented using a mobile operating system framework, such as a general mobile development framework, and stores data temporarily in a local database such as SQLite.

The user carries the terminal during daily life. The user wears a wearable device that measures body activity such as step count and heart rate, and the user optionally captures images of meals or beverages by operating the camera of the terminal. The user may further provide text annotations such as the type of meal, the time of day, or dietary preferences directly into the terminal application. The user may also grant permission for the terminal or the server to access delivery service histories and electronic payment histories stored by external services or by the terminal.

The server receives time-series data that includes body activity data from motion sensors, location data from GPS, intake data from images and user inputs, and transaction data from delivery service histories and electronic payment histories. The server stores this raw data in a persistent storage device, for example a relational database management system such as a general relational database, and an object storage system for large image files. The server organizes the data into tables such as an activity log table, a location log table, a meal image table, and a transaction log table. Each record is associated with a user identifier, a timestamp, and one or more measurement fields.

The server executes a data normalization module implemented in a general-purpose programming language such as Python or a server-side scripting language. The server uses data processing libraries, for example a numerical computation library and a data frame processing library, to convert raw sensor streams into normalized, structured data. The server converts step events into daily total step counts, calculates moving averages of heart rate, and transforms GPS coordinates into approximate distances and movement patterns. The server converts transaction histories into itemized lists of purchased food and beverage items, including quantities, unit prices, and timestamps.

The server executes an image analysis module that processes intake images. In particular embodiments, the server uses a convolutional neural network implemented in a deep learning framework such as TensorFlow or PyTorch. The server configures the convolutional neural network with a plurality of convolutional layers, pooling layers, and fully connected layers. The server trains the neural network in advance using a training dataset comprising labeled food images, where each image is associated with one or more food categories and, optionally, approximate portion sizes. During training, the server applies supervised learning with a loss function such as cross-entropy for classification and mean squared error for portion estimation. The server uses gradient descent-based optimization, such as stochastic gradient descent with momentum or the Adam optimizer, to update neural network weights. The server may also perform data augmentation during training, such as random rotations, scaling, cropping, or color jittering of the food images, to improve the generalization of the model.

The server applies the trained convolutional neural network to each intake image received from the terminal. The server obtains, for each image, one or more predicted food categories and corresponding confidence scores, and optionally an estimated portion size. The server then uses a nutrition database, which maps food categories to nutrition components such as calories, carbohydrates, proteins, and fats, to estimate nutrition values for each intake event.

The server merges these estimates with transaction data when available, so that the portion estimates and menu descriptions from delivery service histories can refine the nutrition estimates. This combination of image-based estimation and transaction-based estimation improves the precision and robustness of intake data, compared to using either source alone. The server derives lifestyle indices and health state indices from the structured data. The server calculates, for each user and for each period such as a day or a week, aggregate metrics including total steps, total walking distance, average daily heart rate during active periods, time spent in sedentary states, number of meals per day, average nutritional balance (for example, ratio of macronutrients), and frequency of late-night meals. The server defines lifestyle indices as feature vectors composed of such metrics, and stores them in a summarized data table. The server may compute additional indices such as variability of activity between weekdays and weekends, or trend indicators comparing the current week to previous weeks.

The server uses a generative AI model to evaluate the lifestyle indices and to generate user advice. In particular embodiments, the server employs a large language model that uses a transformer architecture with multiple self-attention layers and feed-forward layers. The server configures the large language model with a predefined number of layers, attention heads, and hidden units, and uses positional encoding to represent token positions in the input sequence. The server trains or fine-tunes the generative AI model on training data that includes health-related texts, lifestyle recommendations, and example evaluations of fictitious or anonymized users. During training or fine-tuning, the server uses a language modeling loss, such as cross-entropy between predicted token distributions and ground truth tokens, and performs weight updates by stochastic gradient descent or another suitable optimizer. The server may also employ reinforcement learning from human feedback, in which human evaluators rate model outputs and the server uses these ratings to adjust the model parameters so that generated advice better aligns with desired quality and safety criteria.

The server constructs prompt sentences that provide explicit instructions to the generative AI model and embed structured data summaries. The server maintains prompt templates, which are text patterns containing placeholders for user-specific data, task descriptions, and output constraints. The server generates an instance of a prompt sentence by substituting the placeholders with current summary data for a given user and adding explicit instructions about format and scope. For example, the server may generate a prompt sentence of the following form:

“Analyze the following one-week activity and diet data for a single user.

For each day, you are given: total steps, total walking distance (km), active minutes, average heart rate during activity, estimated calorie intake, and brief text summaries of main meals.
1. Evaluate the user's overall health status and lifestyle balance for this week.
2. Identify the main health risks or concerns (for example, insufficient activity, excessive calories, unbalanced nutrition).
3. Summarize the key points in plain English that a non-expert can understand.

Output a concise evaluation (around 300-400 words).

Here is the data:
[structured summary of 7 days' activity and diet data].”

The server also generates prompt sentences for dietary analysis and for advice generation. For example, the server may use a dietary analysis prompt sentence such as:

“Based on the following description of today's meals for one user, estimate:
1. Approximate total calorie intake.
2. Major macronutrient balance (carbohydrates, proteins, fats).
3. Potential issues (for example, too much sugar, too little fiber).
4. Two specific improvement suggestions for the next day's meals.

Use clear, non-technical language.

Here are today's meals:
Breakfast: [text description].
Lunch: [text description].
Dinner: [text description].
Snacks and drinks: [text description].”

The server further generates prompt sentences that specify a virtual instructor persona, for example:

“You are a gentle, encouraging virtual health coach named ‘Alex’.

Using the following one-week summary of steps, activity time, and estimated calorie intake, create personalized advice for the user.

### Constraints:

Do not provide any medical diagnosis.
Focus on practical lifestyle tips about exercise and diet.
Use friendly and supportive language.
Length: about 200-300 words.

### User Data Summary:

[one-week summary of activity and diet features]

Write your advice as if you are speaking directly to the user, using ‘you’.”

The server embeds style information, such as the coach name, tone, and constraint conditions, as explicit tokens in the prompt sentences, which causes the generative AI model to generate outputs consistent with the desired persona. This approach enables the server to control the generative AI model behavior through explicit, machine-generated prompt sentences rather than through manual or static prompt design.

The server uses a dedicated AI interface module to communicate with the generative AI model. The server formats the prompt sentence and any associated structured data into a request message, and transmits the request to an inference endpoint of the generative AI model using a network protocol such as HTTPS. The server specifies parameters such as maximum token length, temperature, and top-k or top-p sampling thresholds, which influence the diversity and determinism of generated text. The server receives the model output as a sequence of tokens, decodes the tokens into natural language text, and stores the raw output in a log. The server then applies parsing rules or a secondary model to segment the text into distinct parts, such as overview, identified issues, and recommendations.

The server converts the parsed advice and evaluation into structured advice data. The server defines data structures, for example a schema with fields such as summary, risks, recommendations, and action items. The server uses pattern detection, keyword matching, or structured output formats specified in the prompt sentence to map segments of the model output to the corresponding fields. This structured representation facilitates indexing, version control, and re-use of advice content. For example, the server can retrieve past advice for comparison or can perform meta-analysis of advice over time.

The server applies an additional layer of processing to enforce safety and quality constraints. The server may use rule-based filters, such as blocking certain medical diagnosis words or enforcing maximum text length, and may also use a secondary discriminator model to detect potentially harmful or off-topic content. If the server detects that the initial output does not meet quality criteria, the server may generate a corrective prompt sentence instructing the generative AI model to rephrase or adjust its output, such as: “Rewrite the following advice in simpler language of approximately 200 words, remove any medical diagnoses, and focus only on lifestyle recommendations.” By iteratively applying such prompts, the server converges on an output that satisfies technical, regulatory, and user experience constraints. The server generates a final advice text that is styled according to the virtual instructor persona and stores it in a database table associated with the user. The server may also derive machine-readable action items, such as target step count or maximum recommended calorie intake per day, which can be used by the terminal application to display progress bars or alerts. The server sends a notification to the terminal indicating that new advice is available. The notification contains a brief summary, while the full advice text is obtained from the server when the user opens the terminal application.

The terminal receives the notification over a push notification service and displays a message to the user. When the user opens the application, the terminal requests the full advice text from the server and renders it on the display. The terminal can show sections such as summary, detailed advice, and specific action items, and can display an avatar or icon representing the virtual instructor persona. The terminal may also allow the user to provide feedback, such as rating the helpfulness of the advice or stating constraints (for example, physical limitations or dietary restrictions), which the server stores and uses in future feature construction and prompt generation.

The server, by structuring the data and the prompt sentences as described, improves computer technology in several ways. First, the server reduces communication load by converting high-frequency raw sensor streams into compressed lifestyle indices before sending data to the generative AI model, rather than transmitting large volumes of unprocessed time-series data. This compression and feature extraction reduce bandwidth usage and improve end-to-end response time. Second, the server improves computational efficiency by standardizing the prompt format and pre-structuring the input data, which allows the generative AI model to focus on reasoning and language generation rather than inferring structure from arbitrary free text. Third, the server improves accuracy and consistency of advice by combining image-based food recognition with transaction-based verification, thereby reducing recognition errors and providing more reliable nutrition estimates. Fourth, the server improves data management by storing both structured lifestyle indices and structured advice data, enabling stable retrieval, comparison, and auditing.

The server, in addition, implements non-conventional processing sequences that differ from manual human work. For example, the server automatically selects and arranges lifestyle indices into prompt sentences in an order and with a level of detail tailored to the generative AI model's training characteristics, which is based on the model's sensitivity to token order and context window limitations. The server may rank features by importance using a feature selection algorithm, such as mutual information or a learned attention weight analysis, and include only top-ranked features in the prompt to stay within a token limit. This selective feature inclusion reduces noise and improves the quality and stability of the model's outputs, which is a machine-specific optimization rather than a straightforward automation of human evaluation.

The server may implement alternative embodiments of the generative AI model and data processing pipeline. In one embodiment, the server hosts the generative AI model locally and executes it on specialized hardware such as graphics processing units (GPUs) or tensor processing units (TPUs). The server uses model quantization and weight pruning techniques to reduce model size and inference time while maintaining acceptable output quality. In another embodiment, the server operates as a client to an external AI service, sending feature summaries and receiving generated advice, wherein the server still controls data structuring, prompt generation, and post-processing. In yet another embodiment, the server runs multiple generative AI models specialized for different tasks, such as one model for health evaluation and another for persona styling, with the server orchestrating the sequence of model calls and combining their outputs.

The terminal can also vary in configuration. In one embodiment, the terminal is a smart watch with limited display size and processing capability, and the server offloads nearly all computation, sending only short, prioritized advice messages. In another embodiment, the terminal is a smartphone or tablet with sufficient power to perform preliminary feature extraction and image pre-processing, such as resizing, cropping, or local anonymization of sensitive image regions, before uploading images to the server, which further reduces bandwidth and server load. In a further embodiment, the terminal runs a local, lightweight model to classify basic activity patterns, and only uploads summarized activity episodes to the server, which further improves power efficiency and privacy.

The user thus interacts with a system in which the server and terminal cooperate to implement a technically specific data acquisition, feature generation, prompt generation, AI inference, and advice distribution pipeline. The use of defined data structures, feature engineering, model architectures, and prompt generation algorithms leads to a concrete improvement in the functioning of the computer system itself, including reduced computation and communication overhead, improved accuracy and consistency of generated outputs, and enhanced manageability of user data and AI outputs, beyond a mere automation of human advice-giving tasks.

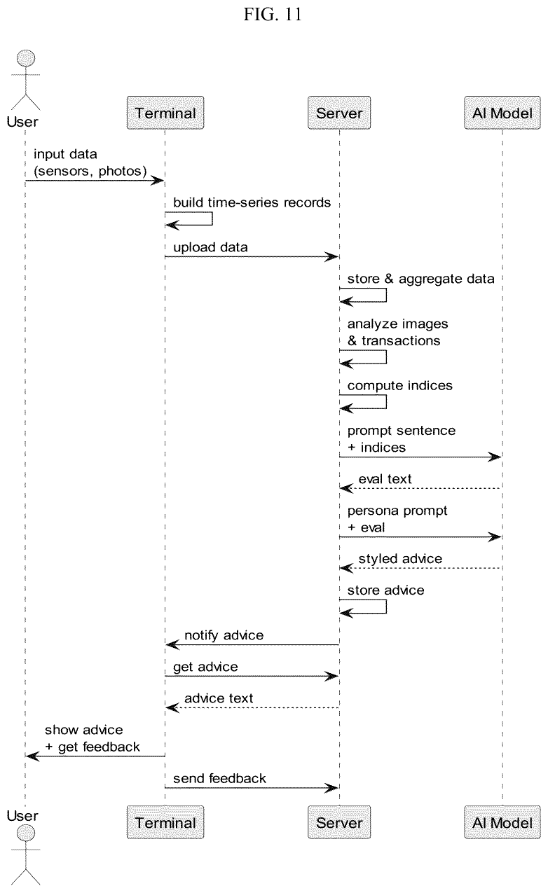

The following describes the processing flow using FIG. 11.

### Step 1:

The terminal acquires raw lifestyle data from the user.

The terminal uses hardware sensors such as an accelerometer, gyroscope, heart-rate sensor, and a GPS receiver to obtain physical activity measurements and location coordinates, and uses a camera to capture intake images. The terminal application receives as input real-time sensor signals, user operations (for example, tapping a “take meal photo” button), and optional text annotations entered by the user. The terminal converts these inputs into time-stamped records containing fields such as step count, activity intensity, heart rate, latitude, longitude, and image file paths. The terminal stores these records in a local storage structure, such as a SQLite table, and queues them for transmission. The output of this step is a set of locally stored, time-series lifestyle data records associated with the user.

### Step 2:

The terminal augments lifestyle data with external service information.

The terminal connects to external service interfaces, such as delivery service APIs and electronic payment service APIs, using secure HTTP requests. The terminal receives as input authentication tokens and user consents, and issues API calls to request order histories and payment histories. The terminal parses the returned structured responses, extracting item names, quantities, prices, merchant identifiers, and timestamps. The terminal merges these transaction entries with local time-series data by aligning timestamps and user identifiers. The terminal outputs enriched lifestyle data records that include both local sensor-derived measurements and external transaction data related to the user's intake behavior.

### Step 3:

The terminal transmits enriched lifestyle data to the server.

The terminal monitors network connectivity and, when a suitable connection is available, selects unsent enriched records from local storage as input. The terminal groups these records into batches and serializes them into a structured format such as JSON, while collecting associated image files. The terminal establishes an encrypted communication channel using TLS and sends the serialized data and images to the server's upload endpoint via HTTPS. The terminal then receives a status response from the server. Based on this response, the terminal marks successfully transmitted records as synchronized and retains or re-queues failed records. The output of this step is a set of enriched lifestyle data records stored on the server and a synchronized state for the corresponding records on the terminal.

### Step 4:

The server receives and validates uploaded lifestyle data.

The server accepts incoming HTTPS requests from the terminal as input. The server terminates TLS, extracts the request body, and parses JSON payloads and attached image files. The server verifies authentication tokens, checks that user identifiers are valid, and confirms that required fields such as timestamps and data types fall within expected ranges. If the validation succeeds, the server writes activity, location, intake, and transaction data into corresponding database tables and stores image files in an object storage system, recording their paths in the database. If errors are detected, the server generates error logs and responds with appropriate error codes. The output of this step is a set of validated and persistently stored lifestyle data entries ready for further processing.

### Step 5:

The server normalizes and aggregates lifestyle data into structured indices.

The server retrieves raw lifestyle records from the database as input, grouped by user and by defined time windows such as days or weeks. The server applies numerical operations using data processing libraries, summing step counts, calculating walking distances from GPS coordinates, computing average heart rates, and determining sedentary intervals by thresholding low-activity periods. For intake data, the server aligns transaction entries with intake images using timestamps and, if available, textual metadata. The server outputs normalized daily or weekly summaries for each user, represented as structured feature vectors or records containing fields such as total steps, total distance, average activity intensity, number of meals, and aggregated transaction-level intake information.

### Step 6:

The server analyzes intake images to estimate food categories and portions.

The server selects intake images and associated metadata (for example, meal time and linked transactions) as input. The server passes these images through a convolutional neural network implemented in a deep learning framework, which has been pre-trained on food images with labeled categories and portion sizes. The neural network performs convolution, pooling, and non-linear activation operations to derive feature maps and outputs class probabilities and portion estimates for each image. The server then maps predicted classes to food categories in a nutrition database and converts portion estimates into approximate quantities. The server produces as output, for each intake event, a set of inferred intake items, estimated portion sizes, and preliminary nutrition values such as calories and macronutrient amounts.

### Step 7:

The server refines nutrition estimates by combining image analysis and transaction data.

The server takes as input the preliminary nutrition estimates from image analysis and the transaction data that include ordered item names and quantities. The server matches intake items and transaction entries using timestamps, location information, and string similarity between recognized food categories and purchase descriptions. The server reconciles discrepancies by applying rules such as selecting transaction-derived portion sizes when confidence in image-based estimation is low, or averaging when both sources are compatible. The server then recalculates nutrition values using refined item and portion information. The output of this step is a more accurate, transaction-verified nutrition profile for each intake event.

### Step 8:

The server computes lifestyle indices and health state indices.

The server uses normalized activity summaries and refined nutrition profiles as input. The server constructs feature vectors that encode metrics such as average daily step count, variance of activity between days, proportion of time spent in moderate-to-vigorous activity, daily total calories, macronutrient ratios, and frequency of late or high-calorie meals. The server may apply statistical operations, such as standardization, trend analysis, and moving averages, to capture temporal patterns. The server then generates lifestyle indices and health state indices, which may be represented as numeric scores or categorized levels (for example, activity level category, nutritional balance category). The output of this step is a set of per-user, per-period indices stored in summary tables.

### Step 9:

The server constructs a task-specific prompt sentence for the generative AI model.

The server retrieves lifestyle indices, health state indices, and relevant user profile information (such as age range and preferences) as input. The server selects a prompt template corresponding to the desired task, such as weekly health evaluation or daily dietary analysis. The server inserts the numerical and categorical indices into designated positions in the template, and appends explicit instructions on what analysis is required and how the output should be formatted. For example, the server may generate the following prompt sentence:

“Analyze the following one-week activity and diet data for a single user.

For each day, you are given: total steps, total walking distance (km), active minutes, average heart rate during activity, estimated calorie intake, and brief text summaries of main meals.
1. Evaluate the user's overall health status and lifestyle balance for this week.
2. Identify the main health risks or concerns (for example, insufficient activity, excessive calories, unbalanced nutrition).
3. Summarize the key points in plain English that a non-expert can understand.

Output a concise evaluation (around 300-400 words).

Here is the data:

[one-week summary of 7 days of activity and diet].”

The output of this step is a fully constructed model input text that includes the prompt sentence and embedded user summary data.

### Step 10:

The server transmits the prompt sentence to the generative AI model and receives evaluation output.

The server provides the constructed model input text and generation parameters (such as maximum token count and sampling settings) as input to an AI interface module. The server sends this information in an API request to a generative AI model endpoint using a network protocol such as HTTPS. The generative AI model, which may be a transformer-based large language model, processes the tokenized prompt and produces output tokens corresponding to an evaluation of the user's lifestyle and identification of health-related issues. The server decodes the tokens back into natural-language text and receives the model's evaluation as output. The server stores this output for subsequent parsing and analysis.

### Step 11:

The server parses and structures the evaluation and advice contents.

The server takes the generative AI model's evaluation text as input. The server applies parsing methods such as pattern matching, rule-based segmentation, or secondary prompts that instruct the model to reformat the evaluation into labeled sections. The server identifies and extracts parts corresponding to summary, risk factors, and preliminary recommendations. The server then stores these parts in a structured form, such as fields in an advice record (for example, summary_text, risks_text, and recommendations_text). The output of this step is structured evaluation and advice contents that can be programmatically manipulated and stored.

### Step 12:

The server generates a persona-specific prompt sentence for virtual instructor styling.

The server uses the structured evaluation and advice contents, together with user-specific persona settings (such as virtual instructor name, tone, and language preference), as input. The server constructs a new prompt sentence that instructs the generative AI model to rewrite or reformat the advice in the style of the virtual instructor. For example, the server may generate:

“You are a gentle, encouraging virtual health coach named ‘Alex’.

Using the following one-week summary and recommendations, create personalized advice for the user.

### Constraints:

Do not provide any medical diagnosis.
Focus on practical lifestyle tips about exercise and diet.
Use friendly and supportive language.
Length: about 200-300 words.

Here are the recommendations:
[structured recommendations_text]”

The server outputs a persona-specific model input text that includes both style instructions and content to be rephrased.

### Step 13:

The server applies the persona-specific prompt to the generative AI model and obtains styled advice text.

The server sends the persona-specific model input text as input to the generative AI model via the AI interface. The generative AI model generates a new text that conforms to the persona's tone and the specified constraints, reusing the substantive content of the original evaluation and recommendations. The server receives this styled advice text and decodes it into plain language. The server may perform a final quality and safety check, such as verifying that no medical diagnoses are present and that the length and politeness constraints are satisfied. The output of this step is a final, persona-styled advice text suitable for delivery to the terminal.

### Step 14:

The server stores the final advice and prepares notification information.

The server takes the styled advice text, associated indices, and metadata such as generation time and data coverage period as input. The server writes a new record into an advice log table, associating the advice with the user and with the underlying lifestyle data used to generate it. The server additionally constructs a short summary string for notification purposes, for example, “Your weekly lifestyle advice is ready.” The server outputs stored advice data and a notification payload that includes at least the user identifier and the summary string.

### Step 15:

The server delivers the notification and advice to the terminal.

The server provides the notification payload as input to a push notification service that supports target device addressing. The server sends a push message to the terminal's registered device token, indicating that new advice is available. When the terminal, in response to user interaction, requests the full advice content, the server receives the request, reads the stored advice entry from the database, and returns the styled advice text and associated structured fields (such as summary and action items). The output of this step is a complete advice package transmitted to the terminal for display.

### Step 16:

The terminal presents the advice and collects user feedback.

The terminal receives the push notification and displays a brief message on the user interface as input to the user. When the user taps the notification, the terminal sends a request to the server and obtains the full advice text and structured elements. The terminal renders the advice on the display, optionally showing the virtual instructor's name and avatar and arranging sections such as overview and recommendations in distinct areas. The terminal may provide interactive elements that allow the user to mark recommendations as completed, rate their usefulness, or specify constraints (for example, “cannot perform high-impact exercise”). The terminal packages this feedback as data records and sends them back to the server. The output of this step is a set of user feedback records that the server can incorporate into future lifestyle indices, prompt sentences, and personalization logic.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health management systems often rely on siloed data sources and static rule-based logic, which limits their ability to provide accurate, timely, and context-aware guidance to a user. In many existing systems, activity data, meal records, and transaction histories are processed independently, if at all, on separate applications or platforms. As a result, the underlying computer system is not configured to efficiently integrate heterogeneous data streams such as sensor data, location data, image data, and electronic transaction data into a unified representation of a user's lifestyle. This leads to fragmented data models, redundant processing, and low-quality recommendations.

Further, known systems that employ machine learning or image recognition typically use such techniques only for isolated tasks, for example, recognizing food items from images, without tightly coupling the results with financial transaction data or longitudinal activity data on a shared time axis. This lack of integrated processing prevents the system from automatically correlating a user's meals with actual purchases, locations, and exercise patterns. Consequently, the computing resources are not effectively utilized to detect patterns such as repeated high-calorie purchases coupled with insufficient physical activity, and the system is unable to generate nuanced, situation-dependent advice.

In addition, generative AI models are increasingly used to provide personalized messages, but conventional approaches often treat the generative AI model as an add-on text generator. They construct prompts in an ad hoc manner, without a structured pipeline that transforms multi-modal lifestyle data into a machine-optimized prompt sentence. This can result in unstable outputs, inconsistent persona behavior, and advice that is only loosely grounded in the underlying data. The absence of a dedicated prompt generation mechanism that systematically encodes health metrics, behavioral patterns, and persona attributes into a prompt forces the processor to perform multiple ad hoc conversions and post-processing steps, increasing latency and computational overhead.

Moreover, many existing systems do not allow user corrections of automatically inferred meal content to be fed back into the core data model and the AI pipeline in a closed loop. Without such feedback integration, the system cannot refine its stored life log data, cannot improve subsequent health evaluation, and cannot adapt the prompt generation process to more accurate user-specific data. This results in a static data representation that fails to exploit the computational advantages of interactive refinement.

There is therefore a need for a computer-implemented system that technically improves the way a processor collects, correlates, and analyzes heterogeneous lifestyle data, that programmatically generates structured prompt sentences for a generative AI model based on integrated life log data and health state evaluation, and that uses user feedback to update the underlying data and regenerate advice. Such a system should improve the internal operation of the computer by defining concrete data structures, processing flows, and model interfaces that reduce fragmentation, enable automated correlation, and stabilize AI-generated guidance, thereby improving the overall efficiency and reliability of the health management computing platform.

The specific processing by the specific processing unit 290 of the data processing device 12 in Application Example 1 is realized by the following means.

The present invention provides a server comprising a processor configured to acquire lifestyle data of a user through at least one terminal device including sensors and healthcare applications, store acquired biological activity data and location information data as structured time-series data, acquire meal image data and transform the meal image data into an input format of an image recognition model, execute inference using a neural-network-based image recognition model to estimate meal attributes and generate normalized meal content data, acquire electronic transaction history data via a transaction-service interface and store the transaction history data in a database, correlate the biological activity data, the location information data, the meal content data, and the electronic transaction history data on a common time axis and location axis to generate integrated life log data, apply evaluation logic to the integrated life log data to compute health state evaluation information including energy balance and activity level, automatically generate a machine-readable prompt sentence that encodes the health state evaluation information, the integrated life log data, and persona profile information, provide the prompt sentence as input to a generative AI model, post-process text output from the generative AI model to derive structured advice and encouragement content associated with a virtual coach persona, transmit presentation control data including the advice and encouragement content to the terminal device, and, in response to user corrections of the meal content data, update the integrated life log data and the health state evaluation information and regenerate the prompt sentence and corresponding AI-generated output. This enables the computer system to more efficiently integrate heterogeneous multi-modal lifestyle data, to perform automated correlation and evaluation on a unified internal data structure, and to generate stable, context-aware, and persona-consistent guidance by systematically converting the evaluated data into optimized prompt sentences for the generative AI model, thereby improving the technical performance, responsiveness, and reliability of the health management functionality implemented by the server.

The term “lifestyle data” refers to information representing daily activities and behaviors of a user, including at least biological activity data, meal-related data, location information data, and electronic transaction history data.

The term “terminal device” refers to an information processing device operated by a user, including at least a portable information processing device equipped with one or more sensors, a display, and network communication functions.

The term “portable terminal device” refers to a terminal device that is wearable or mobile, such as a handheld computing device or a wrist-worn computing device, that includes at least one sensor for detecting movement or physiological parameters and a communication interface for transmitting data to a server.

The term “sensor” refers to a hardware component configured to detect a physical quantity or biological signal, including at least an accelerometer, a gyroscope, a heart rate sensor, a positioning sensor, or a similar sensing element.

The term “healthcare application” refers to a software application running on a terminal device that is configured to acquire, store, or provide access to biological activity data or health-related information of a user through an application programming interface.

The term “biological activity data” refers to data representing physical activity or physiological conditions of a user, including at least step count, movement type, heart rate, and activity duration.

The term “location information data” refers to data representing a geographic position of a user or a terminal device, including at least coordinates, timestamps, and optionally place identifiers or region information.

The term “time-series data” refers to data in which each record includes at least one timestamp and one or more measured or derived values, arranged in chronological order for sequential analysis.

The term “meal image data” refers to digital image data captured by a terminal device that includes at least one food item or meal and is used for image-based meal recognition or nutritional analysis.

The term “image processing program” refers to software configured to perform operations on digital images, including at least resizing, normalizing, format conversion, or other preprocessing necessary to prepare image data for analysis by a machine learning model.

The term “machine learning model” refers to a computational model trained on data to perform pattern recognition or prediction, including at least neural-network-based models used for image recognition, classification, or regression.

The term “image recognition model” refers to a machine learning model configured to receive image data as input and output recognized categories, objects, or attributes, including at least a neural network that estimates food types or meal components from meal image data.

The term “neural network” refers to a machine learning architecture comprising interconnected computational units arranged in layers, configured to process input data and learn mappings from inputs to outputs through parameter optimization.

The term “meal attributes” refers to characteristics of a meal estimated by an image recognition model, including at least a meal type, meal components, and nutritional values such as energy or macronutrient amounts.

The term “meal content data” refers to structured data representing the content of a user's meal, including at least estimated food items, portion sizes, and nutritional values generated or updated by the system.

The term “transaction service” refers to a service that processes electronic payments or purchases and provides electronic transaction data through a programmatic interface.

The term “application programming interface of a transaction service” refers to a programmatic interface provided by a transaction service that allows a processor to send requests and receive electronic transaction history data in a structured format.

The term “electronic transaction history data” refers to data representing electronic payment or purchase events of a user, including at least transaction time, transaction amount, transaction type, and transaction location information.

The term “database” refers to a structured data storage system managed by a database management program, configured to store, index, and retrieve records such as lifestyle data, transaction records, and meal content data.

The term “integrated life log data” refers to a unified data structure in which biological activity data, location information data, meal content data, and electronic transaction history data are correlated and managed along a common time axis and location axis.

The term “timeline format” refers to a representation of integrated life log data in which events and measurements are arranged according to time and optionally associated with corresponding locations.

The term “consumption behavior” refers to patterns of purchasing and using goods or services by a user, including at least frequency of food purchases, types of vendors, and expenditure amounts derived from electronic transaction history data.

The term “evaluation logic” refers to a set of computation rules or algorithms applied to integrated life log data to derive health-related metrics and determine a health state of a user.

The term “health state index” refers to one or more numerical or categorical indicators computed from integrated life log data and used to represent a health condition or health trend of a user.

The term “health state evaluation information” refers to structured information derived from the health state index, including at least an energy balance, an activity level, and a frequency of high-calorie intake.

The term “energy balance” refers to a comparison between estimated energy intake and estimated energy expenditure over a defined time period for a user.

The term “activity level” refers to a quantitative or qualitative measure of a user's physical activity, derived at least from biological activity data such as step count, movement intensity, or duration of exercise.

The term “frequency of high-calorie intake” refers to a metric indicating how often a user consumes meals or food items that exceed a predetermined calorie threshold within a given time period.

The term “prompt sentence” refers to a machine-readable natural language text that encodes health state evaluation information, integrated life log data, and instructions regarding output format, tone, and advice content, and that is provided as input to a generative AI model.

The term “generative AI model” refers to an artificial intelligence model configured to generate text or other content based on input data, including at least a language model that produces natural language text in response to a prompt sentence.

The term “persona profile information” refers to data representing characteristics of a virtual coach, including at least a human image or character attribute, tone, and style of communication selected or set by the user or system.

The term “virtual coach persona” refers to a virtual entity whose behavior and communication style are determined by persona profile information and that is used to present advice content and encouragement content to the user.

The term “advice content” refers to natural language guidance generated based on a user's lifestyle data and health state evaluation, including at least recommendations for meals, exercise, or lifestyle changes.

The term “encouragement content” refers to natural language messages intended to motivate or positively reinforce a user's behavior, generated based on the user's current or historical health state evaluation.

The term “utterance content” refers to formatted advice content and encouragement content structured as one or more messages, sentences, or dialogs to be output from a virtual coach persona.

The term “presentation control data” refers to data that specifies how advice content and encouragement content associated with a virtual coach persona are to be displayed or notified on a terminal device, including at least message text, timing information, and display parameters.

The term “message display function” refers to a function of a terminal device that presents textual or graphical information to a user through a user interface, such as a chat-like view or notification area.

The term “notification function” refers to a function of a terminal device that provides alerts or messages to a user, including at least push notifications, banners, or similar alert mechanisms.

The term “correction input” refers to input provided by a user to modify automatically generated or recognized data, including at least corrections to meal types, food items, or portion sizes in meal content data.

The term “supplementary input” refers to input provided by a user to add information not automatically inferred by the system, including at least additional meal items, subjective conditions, or context about a meal or activity.

The term “scheduler function” refers to a function of the processor or server that triggers execution of predetermined processing tasks at defined time intervals or times, such as periodically acquiring data or updating a health state evaluation.

The term “output data” refers to data transmitted from the server to a terminal device, including at least advice content, encouragement content, and associated presentation control data generated based on a prompt sentence and the generative AI model.

In one embodiment, a server cooperates with one or more terminals operated by a user to implement a lifestyle management system that integrates activity sensing, meal recognition, transaction analysis, and generative guidance. The server includes at least one processor, a memory, a non-volatile storage device, a network interface, and executes a plurality of software modules. The server is connected via a communication network to the terminals, which include at least a smartphone and a wearable device, each having sensors, a display, a user interface, and a network communication module.

A terminal uses a motion sensor, a heart rate sensor, and a positioning sensor to measure biological activity data and location information data of the user. The terminal runs an operating system such as a mobile operating system and a healthcare application that exposes an application programming interface for collecting step counts, activity types, heart rates, and related health metrics. The terminal converts sampled sensor outputs into time-stamped records and stores them in a local buffer. The terminal assigns a device identifier and a user identifier to each record to allow the server to associate records with the correct user.

A terminal acquires meal image data when the user captures a photograph of a meal using the built-in camera. The terminal compresses the image into a digital format, such as JPEG, and associates metadata including a capture timestamp, a location derived from the positioning sensor, and a meal category selected by the user. The terminal sends the time-series biological activity data, the location information data, and the meal image data to the server via an encrypted communication protocol, such as HTTPS, using a representational state transfer interface exposed by the server.

The server receives uploaded data at a network interface, and a communication module validates authentication information and payload formats. The server parses the received biological activity data and location information data, converts them into a normalized internal schema, and stores the records as time-series data in a structured storage, such as a relational database. The server assigns indices on user identifiers and timestamps to enable efficient retrieval and joins. The server stores the meal image data in an object storage region and maintains a reference to the stored image in the database.

The server preprocesses each meal image by using an image processing library, for example running on a central processing unit or a graphics processing unit. The server resizes the image to a fixed resolution, performs color normalization, and converts the image into a numerical tensor representation required by an image recognition model. The server uses a neural network architecture such as a convolutional neural network with multiple convolutional layers, pooling layers, and fully connected layers to perform food recognition. The server loads network parameters stored in the storage device into memory and executes forward propagation on the preprocessed tensor.

The server generates a feature vector in an intermediate layer of the convolutional neural network that encodes high-level features of the meal image, such as shapes, textures, and color distributions. The server feeds this feature vector into a classification layer to compute probability values for a plurality of food categories. The server applies a softmax function to produce normalized probabilities, selects one or more categories above a predetermined probability threshold, and associates these categories with a nutritional database including calorie values, macronutrient composition, and portion size reference values. The server thereby generates structured meal content data including at least food items, estimated servings, and nutritional values.

The server acquires electronic transaction history data by communicating with an external transaction-service system via an application programming interface. The server sends authenticated queries including a user identifier or token and a specified time range. The server receives structured responses, for example in a text-based format such as JSON, and parses transaction records including transaction times, transaction amounts, transaction types, and merchant identifiers. The server optionally resolves merchant identifiers to merchant categories, such as restaurant, grocery, or fast-food outlet, by consulting a merchant classification table.

The server correlates electronic transaction history data with meal content data and location information data. The server compares transaction timestamps with meal capture timestamps and identifies likely matching pairs when the difference in time is within a defined window and the physical distance between the transaction location and the meal location is below a threshold. The server links the meal image, the recognized meal content data, and the associated transaction record into a unified event. The server thereby reduces ambiguity in food recognition by using both visual recognition and transaction context, which improves the accuracy of calorie estimation compared to a purely image-based system.

The server defines an internal data structure referred to as integrated life log data. The integrated life log data associates, for each time unit such as a minute or an hour, biological activity metrics, location information, meal content data, and transaction-derived attributes. The server stores the integrated life log data in a database table or in a columnar datastore optimized for time-series analysis. The server uses indices and partitioning schemes to enable efficient range queries over time windows and to reduce input/output overhead when retrieving multi-day histories.

The server applies evaluation logic to the integrated life log data to derive health state evaluation information. The server computes an energy balance by summing estimated calorie intake from meal content data and subtracting estimated energy expenditure based on activity level, weight, and other parameters. The server computes an activity level metric using step counts, intensities inferred from accelerometer-derived features, and durations of moderate-to-vigorous activities. The server calculates a frequency of high-calorie intake by counting events in which estimated meal energy exceeds a defined threshold. The server encodes these metrics into a health state index that may include both numerical scores and categorical labels such as “high calorie day,” “low activity day,” or “balanced day.” The server implements the evaluation logic using algorithms that operate on the integrated life log data instead of isolated data sources. This non-conventional data integration and evaluation pipeline reduces data redundancy and avoids repeated parsing of raw logs by maintaining a single, normalized representation of a user's lifestyle. As a result, the server reduces memory usage and improves processing speed when recomputing health state evaluation information in response to new data or user corrections.

The server generates a prompt sentence for input to a generative AI model by transforming the health state evaluation information and relevant portions of the integrated life log data into a natural language representation. The server constructs the prompt sentence using a template-based and rule-based mechanism executed by the processor. The server first selects salient features from the integrated life log data, such as total steps, total calories, recent high-calorie meals, and recurring vendor categories. The server then inserts these features into parameterized sentence patterns. The server appends explicit instructions regarding desired output format, maximum length, tone, and content focus.

The server further incorporates persona profile information into the prompt sentence. The server retrieves persona attributes, such as “supportive coach,” “brief and practical,” or “detailed educational,” from a persona profile table, and encodes these attributes in the text as explicit instructions to the generative AI model. For example, the server may construct a prompt sentence such as:

“Based on the user's activity and meals today (high-calorie fast-food lunch, 4,000 steps so far, little vigorous exercise), please propose a healthy dinner plan that reduces total daily calorie intake and suggest an appropriate amount of evening exercise. Respond as a friendly virtual fitness coach, using concise and practical advice.”

Alternatively, the server may use a prompt sentence such as:

“The user ate a high-calorie burger-and-fries lunch and has walked only 3,500 steps by 5 p.m. Using this information, provide specific, practical advice for a low-calorie dinner and a moderate exercise routine that can be completed in 30 minutes. Use an encouraging tone and avoid technical jargon.”

The server then transmits the prompt sentence to a generative AI model hosted either on the same server or on a remote computing system. The generative AI model is, for example, a large-scale neural network language model trained on sequences of tokens. The model includes multiple transformer layers with attention mechanisms and is parameterized by a plurality of weights and biases that have been learned through training. During training, the model minimizes a loss function such as cross-entropy between predicted tokens and actual tokens. The model parameters are updated by an optimization algorithm, such as stochastic gradient descent with adaptive learning rates.

The server formats the prompt sentence as a sequence of tokens compatible with the generative AI model's tokenizer. The server sets control parameters such as sampling temperature, maximum token length, and penalty terms for repetition. The generative AI model then computes attention-weighted representations in each layer and outputs a sequence of tokens representing a candidate response. The server decodes these tokens into text and receives the generated text data as a response.

The server post-processes the text data by segmenting the response into logical components such as recommended meals, exercise suggestions, and motivational phrases. The server filters content to remove redundant or non-compliant parts, and enforces constraints such as maximum length or language. The server maps segments into internal fields labeled as advice content and encouragement content. The server associates this content with a virtual coach persona by tagging the content with persona identifiers and display parameters.

The server then generates presentation control data that specifies, for each message, a message identifier, the associated persona attributes, the display timing, and the display format. The server sends the presentation control data to the terminal over the network. The terminal receives the presentation control data and renders the advice and encouragement on a graphical user interface, such as a chat view or a notification banner. The terminal may also display associated quantitative metrics, including time-series graphs of steps or calorie balance, thereby enabling the user to visually confirm the relation between numerical metrics and textual guidance.

The user may correct or supplement automatically generated meal content data through an interface on the terminal. The user can change food item names, adjust portion sizes, or add missed items. The terminal sends correction input and supplementary input to the server. The server updates the corresponding meal content records and recalculates integrated life log data and health state evaluation information. The server then regenerates a prompt sentence based on the updated data and obtains updated advice from the generative AI model. As a result, the system forms a closed loop in which user feedback is incorporated into the core data structures and computational pipeline, progressively improving accuracy.

The system improves computer technology by defining specific internal data structures and processing flows that are not conventional in typical health or wellness applications. The integrated life log data allows the server to execute a single pass over combined sensor, image, and transaction information instead of multiple passes over disparate logs. This reduces database query count and network usage, thereby reducing latency. The structured prompt generation mechanism also reduces computational overhead in the generative AI model by providing more informative and constrained inputs, which leads to shorter and more relevant outputs and lower token usage.

In addition, the combined use of food recognition from images and transaction-based matching improves the reliability of meal identification compared to relying on single data sources. The server's correlation algorithm uses non-trivial rules that combine time window matching, spatial distance thresholds, and merchant-category mapping, which a human operator would not feasibly apply in real time to continuous data streams. This multi-source fusion reduces misclassification errors in calorie estimation and improves the overall accuracy of health state evaluation.

The generative AI model is not used as a generic text generator. Instead, the server uses a specialized prompt generation module that enforces a structured representation of health metrics and persona attributes. This architecture modifies the way the computer internally represents and processes lifestyle data. By mapping numerical health metrics, contextual event sequences, and persona parameters into a standardized prompt syntax, the server enables deterministic control over content generation. This approach stabilizes outputs across sessions and users, which represents an improvement in the technical functioning of the generative AI pipeline.

The server also reduces communication load by scheduling periodic synchronization windows and batching uploads and responses. The scheduler function in the server determines appropriate intervals for acquiring new sensor data and transaction data, updating integrated life log data, computing new health state evaluation information, and generating prompt sentences. The server avoids continuous polling and instead schedules tasks at optimized intervals based on data variability and user interaction frequency. This design reduces unnecessary network traffic and lowers power consumption on the terminal.

In alternative embodiments, the server may employ different neural network architectures for image recognition, such as residual networks or vision transformers, and may use different feature extraction methods. The generative AI model may be fine-tuned on domain-specific corpora of health advice to improve alignment with the system's objectives. The evaluation logic may incorporate additional features, such as sleep patterns or stress indicators, and may use models such as gradient boosting machines or recurrent neural networks to predict health risks. The core architecture of integrated life log data, structured prompt generation, and closed-loop feedback with a generative AI model, however, remains consistent across such variations.

In another embodiment, the terminal may perform part of the preprocessing or evaluation locally to further reduce server load and latency. The terminal may execute lightweight models for preliminary food recognition and send intermediate representations instead of raw images. The server then refines the recognition using more complex models. This division of labor between the terminal and the server further optimizes computational efficiency and communication bandwidth.

Through these configurations, the server and the terminals cooperate to implement a technical solution that goes beyond a mere automation of human manual analysis. The system introduces non-conventional data integration, evaluation, and prompt generation mechanisms that modify the way computer components process heterogeneous lifestyle data and interact with a generative AI model. As a result, the system achieves improved processing speed, higher accuracy of health-related inferences, reduced communication overhead, and more consistent AI-generated guidance compared to conventional systems.

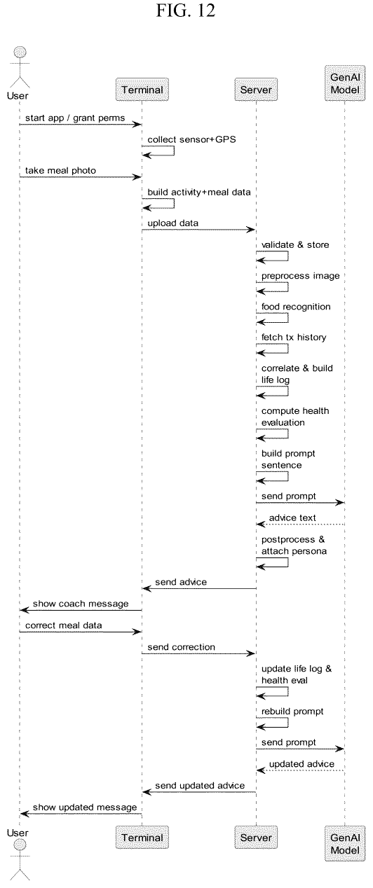

The following describes the processing flow using FIG. 12.

### Step 1:

The terminal initializes data acquisition for the user.

The terminal receives, from the user, permissions to access motion sensors, location services, and healthcare application interfaces. As input, the terminal obtains sensor access settings, user identifiers, and authentication tokens for a healthcare application. The terminal writes these parameters into secure storage and initializes background services that periodically read sensor outputs. Based on these inputs, the terminal outputs a configuration state that specifies sampling intervals, enabled sensors, and server endpoints for later data transmission.

### Step 2:

The terminal collects biological activity data and location information.

The terminal uses motion sensors and heart rate sensors to sample raw signals at defined intervals. As input, the terminal takes the sensor readings (e.g., acceleration values, heart rate values) and timestamps from the system clock, and it calls a healthcare application interface to obtain derived metrics such as step counts and activity types. The terminal combines these inputs with GPS coordinates from the positioning sensor to create structured records. The terminal performs data processing by aggregating, for each time unit, raw samples into summary values such as total steps, average heart rate, and dominant activity type. The terminal outputs a time-series buffer of biological activity data and location information tagged with the user identifier.

### Step 3:

The terminal acquires meal image data and related metadata.

The terminal activates the camera application when the user starts a meal logging operation. As input, the terminal receives raw image frames from the camera, the capture timestamp, the current location, and a meal category selected by the user (e.g., breakfast, lunch, dinner, snack). The terminal compresses the selected frame into a digital image file, attaches metadata fields (time, location, category, device ID, user ID), and stores the image temporarily in local memory. The terminal outputs a meal image object consisting of the compressed file and associated metadata ready to be uploaded.

### Step 4:

The terminal transmits collected data to the server.

The terminal opens a network connection to the server and prepares a request body. As input, the terminal uses the buffered biological activity data, the location information data, and the meal image object, along with authentication tokens. The terminal serializes time-series data into a structured text format and attaches the meal image file using a multipart request. The terminal performs data packaging and encryption via a secure transport protocol, and then sends the request to a server endpoint. The terminal outputs a transmitted data packet and waits for an acknowledgment from the server, clearing the corresponding local buffers upon successful confirmation.

### Step 5:

The server receives and validates lifestyle data.

The server accepts incoming requests at a communication interface. As input, the server receives the data packet including biological activity records, location information, and meal image data, along with authentication headers. The server parses the request, validates digital signatures or tokens, checks schema conformity and payload size, and logs request metadata. The server performs data validation operations such as type checking, timestamp consistency checking, and user ID verification. The server outputs normalized in-memory objects representing activity data, location data, and meal images that are ready for storage and analysis.

### Step 6

The server stores raw data and constructs time-series records.

The server writes validated lifestyle data into persistent storage. As input, the server uses the normalized activity records and location records with timestamps and user identifiers. The server maps fields onto predefined database schemas, converts timestamps to a unified time zone, and assigns internal sequence identifiers. The server performs indexing operations on user ID and time fields to accelerate future queries. The server outputs stored time-series records in a database and stored meal image files in an object storage region, each referenced by unique keys.

### Step 7:

The server preprocesses meal image data for recognition.

The server retrieves a stored meal image file based on a reference key. As input, the server takes the original image file and associated metadata (resolution, orientation). The server uses an image processing library to resize the image to a fixed dimension, normalize pixel intensities, and adjust color channels. The server then converts the pixel array into a numerical tensor with a specific shape expected by an image recognition model. The server outputs a preprocessed image tensor and updated metadata (e.g., new dimensions, scaling parameters) for use in the next step.

### Step 8:

The server executes food recognition using an image recognition model.

The server loads a trained neural network into memory, such as a convolutional network with convolution, pooling, and fully-connected layers. As input, the server uses the preprocessed image tensor. The server performs forward propagation, computing feature maps in successive layers and applying non-linear activation functions. The server then applies a classification layer with a softmax function to obtain probability scores over a predefined set of food categories. Using these probabilities, the server selects one or more food categories that exceed a threshold and retrieves nutritional profiles (calories, macronutrients, typical serving sizes) from a nutritional database. The server outputs structured meal content data that includes recognized items, estimated portion sizes, and computed nutritional values.

### Step 9:

The server acquires electronic transaction history data.

The server communicates with an external transaction-service interface. As input, the server uses a user-linked token, a requested time period, and optional filters such as merchant category. The server sends a query to the transaction-service system and receives structured transaction records that include transaction timestamps, amounts, merchant identifiers, and transaction locations. The server parses the returned text data, converts fields to internal formats, and optionally maps merchant identifiers to merchant categories using a merchant classification table. The server outputs stored transaction history entries linked to the user in the internal database.

### Step 10:

The server correlates transaction records with meals and locations.

The server retrieves, as input, the meal content data, meal capture timestamps, meal locations, and relevant transaction records for the same period. The server executes a correlation algorithm that compares timestamps and computes temporal differences between meals and transactions, and spatial distances between meal locations and transaction locations. The server applies thresholds on time and distance, and uses merchant category filters to identify probable pairs where a transaction corresponds to a logged meal. The server then associates matching transactions with the meal content data, updating entries with additional information such as vendor type and actual purchase amount. The server outputs enriched meal events that combine image-based recognition results and transaction-based context.

### Step 11:

The server constructs integrated life log data.

The server gathers, as input, biological activity records, location information, enriched meal events, and transaction history records for a given time span. The server aligns these data on a common time axis by assigning each event or measurement to discrete time intervals (for example, every 5 minutes or every 1 hour). The server interpolates or aggregates values within each interval, computing derived metrics such as step count per interval, average heart rate, location region, and calorie intake per interval. The server stores these combined records in a dedicated data structure referred to as integrated life log data. The server outputs a unified timeline where each time unit contains linked activity features, meal features, and spending features for the user.

### Step 12:

The server evaluates a health state based on the integrated life log data.

The server uses the integrated life log data as input to an evaluation module. The server calculates total daily steps, total estimated calorie intake, total estimated energy expenditure, and counts of high-calorie meals detected by thresholding meal energy values. The server computes an energy balance as the difference between intake and expenditure and determines an activity level by comparing total steps and exercise intensity to predefined reference values. The server also calculates a frequency of high-calorie intake by counting qualifying meal events within the day. The server encodes these results into health state evaluation information, including numerical indices and qualitative labels such as “excess intake, low activity.” The server outputs the health state evaluation information as a structured object linked to the user and date.

### Step 13:

The server generates a prompt sentence for a generative AI model.

The server constructs, as input, the health state evaluation information and selected elements of the integrated life log data, such as descriptions of recent meals and activity summaries. The server also retrieves persona profile information that defines a virtual coach style. The server formats these inputs using a template mechanism that inserts concrete metric values and persona attributes into natural language patterns. The server adds explicit instructions about response length, tone, and output structure. For example, the server may generate a prompt sentence:

“Based on the user's activity and meals today (high-calorie fast-food lunch, 4,000 steps so far, little vigorous exercise), please propose a healthy dinner plan that reduces total daily calorie intake and suggest an appropriate amount of evening exercise. Respond as a friendly virtual fitness coach using concise, practical advice.”

The server outputs a finalized prompt sentence string that is ready for submission to the generative AI model.

### Step 14:

The server obtains advice content and encouragement content from the generative AI model.

The server submits the prompt sentence as input to a generative AI model interface, along with control parameters such as maximum response length and temperature. The generative AI model processes the tokenized prompt and returns a generated text sequence. The server receives this generated text and performs parsing operations to separate recommendations for meals, suggestions for physical activity, and motivational language. The server filters redundant phrases and ensures compliance with content policies. The server outputs structured advice content (e.g., specific meal and exercise suggestions) and encouragement content (e.g., supportive messages).

### Step 15:

The server associates generated content with a virtual coach persona and prepares presentation control data.

The server uses persona profile information as input together with the advice content and encouragement content. The server assigns persona-specific attributes such as a display name, avatar reference, and tone indicators to each piece of content. The server constructs presentation control data including message text, scheduling information, and user interface hints (for example, display as a chat bubble or as a notification). The server outputs a presentation control data object that encapsulates all information required by the terminal to render the virtual coach's messages to the user.

### Step 16:

The terminal presents advice and encouragement to the user.

The terminal receives presentation control data as input from the server. The terminal parses the data, maps persona identifiers to locally stored visual assets (such as avatar images), and formats the advice content and encouragement content into user interface components. The terminal displays the virtual coach messages in a chronological view and, optionally, overlays or aligns them with charts showing steps and calorie trends. The terminal uses built-in notification mechanisms to alert the user when new guidance becomes available. The terminal outputs visual and audible signals that deliver the generated guidance to the user.

### Step 17:

The user reviews content and provides correction or supplementary input for meals.

The user inspects the displayed meal details and suggestions. As input, the user selects a logged meal on the terminal and edits recognized items or portion sizes, or adds missing food items. The terminal records the modified values and any additional text comments. The terminal performs validation on user entries, such as checking that portion sizes are numeric, and sends the corrected and supplementary meal data to the server. The terminal outputs an update request containing the modified meal content data.

### Step 18:

The server updates integrated life log data and regenerates guidance.

The server receives, as input, the update request containing corrected or supplementary meal content data. The server recalculates nutritional values for the affected meal events and updates the integrated life log data accordingly, recomputing per-interval and daily calorie totals. The server runs the evaluation logic again to update the health state evaluation information. Based on the updated results, the server generates a new prompt sentence and calls the generative AI model as in the previous steps. The server outputs updated advice content and encouragement content, which are transmitted to the terminal in updated presentation control data, thereby closing the feedback loop and improving the precision of subsequent recommendations.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented coaching and wellness systems typically rely on static rule sets, fixed user segments, or manually authored templates to generate exercise and diet advice. In such systems, a processor often aggregates sensor data and logs but then applies simple threshold logic or pre-defined decision trees to determine which messages to show. As a result, the same or similar advice is repeatedly presented to users with heterogeneous personalities, preferences, and lifestyles, which leads to low engagement, rapid user fatigue, and poor long-term adherence to health programs.

Furthermore, although recent systems may integrate machine learning components or generative models, they frequently treat the generative model as a black box that is driven by coarse user attributes or generic prompts. In these implementations, the processor tends to map raw user data directly to generic model inputs, without robust feature engineering or systematic management of prompt sentences. This often causes instability in generated outputs, difficulty in controlling the tone and content of virtual instructors, and an inability to consistently adapt the system to the evolving behavior and feedback of individual users. In addition, feedback from users—such as satisfaction scores, comments on advice, or logs of task completion—is often stored only as historical data for reporting or business analytics, and is not tightly integrated into the computational loop that governs how prompts are constructed and how the generative model is used. Consequently, the underlying computer system cannot effectively refine its internal representations or update its generative behavior based on long-term, fine-grained feedback, which limits the technical capability of the system to deliver increasingly accurate and user-appropriate outputs over time.

Thus, there is a need for an improved computer-implemented system and method in which a processor: (i) systematically converts heterogeneous daily-life information into structured numerical feature vectors; (ii) constructs prompt sentences that are explicitly conditioned on these feature vectors; (iii) drives a generative artificial intelligence model with such prompt sentences to generate virtual instructor profiles and advice messages; and (iv) closes the loop by using accumulated user feedback to update at least one of the generative model and the prompt-generation process. Addressing these issues would improve the functioning of the computer system itself, including more efficient use of model capacity, more controllable and stable generation behavior, and enhanced personalization in real time or near real time.

The specific processing by the specific processing unit 290 of the data processing device 12 in Example 2 is realized by the following means.

The present invention provides a server comprising a processor configured to acquire user daily-life information from a portable information terminal via a communication network; preprocess the user daily-life information by using a tabular data processing function to generate feature information indicating at least one of personality, preference, lifestyle, exercise status, and dietary status of a user; convert the feature information into a numerical feature vector by using a machine-learning feature transformation function and store the numerical feature vector in association with the user; generate a prompt sentence in natural language, based on the numerical feature vector, summarizing at least one of the personality, preference, lifestyle, exercise status, and dietary status of the user; execute a generative artificial intelligence model with the prompt sentence as an input to generate profile information of a virtual instructor and one or more advice messages to be provided to the user; transmit the profile information and the one or more advice messages to the portable information terminal for presentation to the user; acquire evaluation information and feedback information from the user regarding at least one of the virtual instructor and the one or more advice messages; and update at least one of the generative artificial intelligence model and a process of generating the prompt sentence based on a correspondence among the evaluation information, the feedback information, the numerical feature vector, and the prompt sentence. This enables a technical improvement in computer-based personalization and content generation, by allowing the server to construct model-ready numerical representations and prompt sentences in a controlled and repeatable manner, to generate virtual instructor outputs that are dynamically adapted to rich user profiles, and to iteratively refine the underlying generative behavior of the system using accumulated feedback, thereby enhancing stability, accuracy, and efficiency of the overall computing process.

The term “processor” refers to a hardware information processing unit, or a combination of such units, configured to execute instructions to perform one or more of data acquisition, data preprocessing, feature extraction, prompt generation, model inference, result transmission, and model updating.

The term “user daily-life information” refers to information relating to a user's everyday activities and conditions, including at least one of personality attributes, preferences, lifestyle patterns, exercise status, and dietary status, which is input by the user or collected via a portable information terminal.

The term “portable information terminal” refers to an electronic device that a user can carry, including at least one of a smartphone, a tablet, or a wearable device, which executes an information processing program, interacts with the user through a user interface, and communicates with a server via a communication network.

The term “communication network” refers to a wired or wireless communication infrastructure, including at least one of a local area network, a wide area network, and the Internet, through which data is transmitted between the portable information terminal and the server.

The term “information processing program” refers to software executed on the portable information terminal that is configured to present input screens to the user, receive user daily-life information, display virtual instructor information and advice messages, collect user feedback, and communicate with the server.

The term “tabular data processing function” refers to a software-based data manipulation capability configured to operate on data structures having rows and columns, including at least one of cleaning, normalizing, aggregating, and transforming user daily-life information into structured feature information.

The term “feature information” refers to structured data derived from user daily-life information, indicating at least one of the personality, preference, lifestyle, exercise status, and dietary status of the user in a form suitable for further numerical processing.

The term “machine-learning feature transformation function” refers to a computational function or module configured to convert feature information into numerical feature vectors, including at least one of encoding categorical variables, scaling or normalizing numerical values, and transforming text data into numerical representations.

The term “numerical feature vector” refers to an ordered set of numerical values that collectively represent feature information of a user, and that is suitable for input to a machine learning model or a generative artificial intelligence model.

The term “prompt sentence” refers to a sequence of natural language text that is generated based on the numerical feature vector and that summarizes at least one of the personality, preference, lifestyle, exercise status, and dietary status of the user, and that is supplied as input to a generative artificial intelligence model.

The term “generative artificial intelligence model” refers to a machine learning model configured to receive a prompt sentence and optionally other inputs, and to generate output data including at least one of profile information of a virtual instructor and advice messages, by performing generative inference.

The term “virtual instructor” refers to a virtual entity defined by profile information and configured to provide advice and encouragement to the user regarding exercise and diet, the behavior and messages of which are generated by the generative artificial intelligence model.

The term “profile information of a virtual instructor” refers to information that defines characteristics of the virtual instructor, including at least one of name, personality, communication style, and coaching style, and that is generated by the generative artificial intelligence model based on the prompt sentence.

The term “advice message” refers to text generated by the generative artificial intelligence model that provides the user with guidance, recommendations, or encouragement related to at least one of exercise and diet, and that is output in association with the virtual instructor.

The term “display unit” refers to a hardware component of the portable information terminal configured to visually present information to the user, including at least one of a liquid crystal display, an organic light-emitting display, or another graphical output device.

The term “evaluation information” refers to data indicating the user's assessment of at least one of the virtual instructor and the advice messages, including at least one of ratings, scores, or selection of evaluation options.

The term “feedback information” refers to user-supplied information regarding the virtual instructor or advice messages, including at least one of free-text comments, suggestions, or qualitative responses, which is used to assess and improve system performance.

The term “visual display object” refers to a graphical element generated based on the profile information of the virtual instructor, representing at least a character or avatar of the virtual instructor, and displayed on a user interface of the portable information terminal.

The term “task completion information” refers to information indicating whether and how the user has executed actions suggested by the virtual instructor, including at least one of completion flags, timestamps, and quantitative achievement data.

The term “behavior history information” refers to historical data regarding the user's actions and interactions with the system, including at least one of exercise logs, diet logs, interaction logs with the virtual instructor, and use history of the application.

The term “generation accuracy” refers to the degree to which the profile information of the virtual instructor and the advice messages generated by the generative artificial intelligence model appropriately reflect the user's characteristics and preferences and satisfy user expectations, as evaluated based on evaluation information and feedback information.

In one embodiment, a server, a terminal, and a user cooperate to implement the invention.

The server includes at least one processor, a memory, a communication interface, and a storage device. The processor executes server-side application software, a data processing framework, and a generative AI model. The memory stores executable instructions and intermediate data structures such as tabular data, feature vectors, and prompt sentences. The storage device stores a user database, a log database, a model parameter store, and configuration data for feature transformation and prompt generation.

The terminal includes at least one processor, a memory, a communication interface, a display unit, and an input unit such as a touch panel. The terminal executes an information processing program that presents graphical user interfaces, acquires user daily-life information, transmits the information to the server, displays a virtual instructor and advice messages, and collects user evaluation and feedback.

The user operates the terminal to enter daily-life information, to view profile information and advice messages of a virtual instructor, and to provide evaluation information and feedback information.

### 1. Hardware and Software Configuration

The server uses a general-purpose computing platform, such as a rack-mounted server or a virtual machine in a data center. The server runs an operating system and an application framework. The server executes a data processing environment that includes, for example, a Python runtime, a tabular data processing library such as a DataFrame-based library (e.g., a library of the Pandas type), and a machine-learning library such as a feature transformation toolkit (e.g., a toolkit of the Scikit-learn type). The server executes a generative AI framework such as a tensor-based deep learning framework (e.g., a framework of the TensorFlow type or the PyTorch type) to implement a generative AI model. The server can be equipped with a graphics processing unit to accelerate matrix operations and neural network inference.

The terminal uses a portable electronic device such as a smartphone, a tablet, or a wearable computing device. The terminal runs a mobile operating system and a user-interface framework, such as a native UI toolkit or a cross-platform UI toolkit. The terminal executes an application that includes modules for input forms, data validation, HTTP communication, and local storage (e.g., a relational storage component of the SQLite type or a key-value storage component). The terminal includes sensors such as an accelerometer, a gyroscope, a heart rate sensor, or a location sensor, and the application may obtain sensor data via an operating system API and incorporate such data into user daily-life information.

### 2. Data Structures and Internal Representations

The server uses a tabular data structure to store user daily-life information. For example, the server constructs a table where each row corresponds to a record for a particular user and time, and each column corresponds to an attribute such as personality answer, preference category, exercise duration, exercise intensity, meal description, calorie estimate, and timestamp.

The server uses a feature information structure that is derived from the tabular data. The feature information includes normalized numeric attributes (e.g., weekly exercise minutes, average daily steps, average daily calorie intake), encoded categorical attributes (e.g., personality types, preference clusters, lifestyle segments), and processed text attributes (e.g., bag-of-words or embedding-based representation of user comments). The server then converts the feature information into a numerical feature vector represented as an array or tensor of real numbers.

The server uses a prompt sentence representation as a text string. The prompt sentence consists of one or more sentences in natural language that describe the user's personality, preference, lifestyle, exercise status, and dietary status. The prompt sentence may include explicit fields and constraints for the generative AI model, such as required output format, number of messages, or style of the virtual instructor.

The server uses a profile information structure for the virtual instructor. The profile information includes attributes such as a name field, a personality description field, a communication style field, and a coaching style field. The profile information also includes one or more example advice messages that the virtual instructor is expected to output. The terminal uses an internal data structure to represent the virtual instructor and advice messages. The terminal may map the virtual instructor personality type to a particular visual asset identifier, and may associate each advice message with a timestamp and context.

### 3. Generative AI Model Architecture and Training

The server implements the generative AI model as a neural network. In one embodiment, the server uses a transformer-based language model. The server represents text as token sequences using a tokenizer that maps words or subword units to integer indices. The server defines an embedding layer that maps each token index to a dense vector. The server applies multiple self-attention layers that include multi-head attention mechanisms, feedforward sublayers, and normalization layers. The server defines an output layer that maps hidden vectors to probability distributions over tokens, and the server generates text by sampling or selecting tokens based on the output probabilities.

The server trains or fine-tunes the generative AI model on a training dataset that includes pairs of (prompt sentence, target output text). The target output text describes virtual instructor profiles and advice messages that are suitable for various user segments. During training, the server uses a loss function such as a negative log-likelihood or cross-entropy between the predicted token distribution and the target token sequence. The server updates model weights by performing gradient-based optimization such as stochastic gradient descent or an adaptive method. The server may employ data augmentation techniques such as paraphrasing, synonym replacement, or slight numeric perturbation of user feature descriptions to improve robustness.

The server can also use feedback-aware fine-tuning. The server collects pairs of (prompt sentence, generated output, evaluation score, feedback text) and constructs a secondary dataset where highly rated outputs are weighted more strongly. The server may implement a ranking loss or a reward-weighted loss function so that, during fine-tuning, generated outputs that received higher evaluation information from users contribute more to weight updates. This feedback-conditioned training enables the model to adapt its generation behavior to real-world user preferences, thus improving accuracy and user satisfaction.

### 4. Feature Transformation and Prompt Generation

The server uses a dedicated feature transformation module implemented using a machine-learning feature transformation library. The server applies algorithms such as one-hot encoding for categorical features, standardization for continuous features, and text vectorization for short comments. For example, the server may use a one-hot encoder for discrete personality types, a scaler for exercise duration and step counts, and a frequency-based vectorizer for brief preference descriptions.

The server uses a feature selection or dimensionality-reduction process to avoid redundant features and reduce computational load. The server may apply a principal component analysis or another projection technique to the numerical feature vector, thus lowering dimensionality while retaining key variations relevant to user behavior. This dimensionality reduction contributes to reduced computation time during model inference and helps mitigate overfitting.

The server uses a prompt construction algorithm that follows rules that differ from simple human-written templates. For example, the server selects only those features that exceed certain thresholds, such as “weekly exercise minutes above a pre-defined level,” and the server maps numeric ranges to linguistic descriptions such as “low,” “moderate,” or “high” activity level according to predetermined rules. The server sorts feature descriptions by importance, defined by a weighting vector derived from feature importance analysis. The server then concatenates these ordered descriptions into a structured text block, ensuring a consistent format that is optimized for the generative AI model.

In one example, the server generates a prompt sentence as follows:

“User personality: active and extroverted.

Exercise: runs 3 times per week (about 40 minutes each), enjoys outdoor running.

Diet: balanced, goal is weight maintenance.

Generate a personalized virtual trainer profile that matches this user.

The trainer should be energetic and motivating, with short and practical advice.

Output trainer name, short personality description, coaching style, and 3 example messages for exercise and diet.”

In another example, the server generates a more compact prompt sentence:

“The user has an active personality and a high exercise volume.

Generate a virtual trainer that matches this user.

The trainer should have a lively and positive character, give practical running tips, and provide short, motivational messages that help the user continue exercising.

Output a virtual trainer name, a brief character description, a communication style, and example advice messages.”

The server's rule-based and feature-weighted prompt construction is tailored to the generative AI model and is not a simple manual script. This structured mapping from numerical feature vectors to prompt sentences improves the stability and controllability of model outputs and reduces the probability of irrelevant or inconsistent responses.

### 5. Virtual Instructor Generation and Visualization

The server executes the generative AI model using the constructed prompt sentence as input. The server performs tokenization of the prompt sentence, passes the token sequence through the neural network, and obtains a sequence of output tokens representing the textual description of the virtual instructor profile and advice messages. The server can control output length and style by setting decoding parameters such as temperature, top-k or top-p sampling thresholds, and maximum token length.

The server parses the generated text into components, using explicit markers or pattern matching, and maps the components into a profile information structure. For example, the server identifies a line starting with “Name:” as the name field, and lines starting with ordinal numbers as individual advice messages. The server checks that required fields exist and, if fields are missing, the server may regenerate or repair the output by invoking the model with a refined prompt sentence that includes additional constraints.

The terminal receives the structured profile information and advice messages and selects a visual asset based on the virtual instructor personality type. For example, an “energetic” type may be associated with bright color schemes and dynamic posture, while a “calm” type may be associated with subdued colors and relaxed posture. The terminal composes a visual display object comprising the avatar image, the instructor name, and a short textual description. The terminal then displays the visual display object together with the advice messages on the display unit.

The user views the virtual instructor and reads the advice messages. The user may tap actionable buttons to mark advice as completed, to request additional advice, or to access a different type of content. The terminal records these interactions and may send completion flags and timestamps to the server as task completion information and behavior history information.

### 6. Feedback Collection and Model Updating

The terminal displays interfaces that accept evaluation information and feedback information from the user. The user can assign a rating score to the virtual instructor or a specific advice message and can enter textual feedback such as “The trainer is too strict” or “I would like more stretching exercises.” The terminal packages this information with identifiers of the relevant virtual instructor profile and advice messages, and sends the package to the server. The server stores evaluation information and feedback information in a feedback database along with associated numerical feature vectors and prompt sentences. The server periodically retrieves the feedback records and performs statistical analysis. The server aggregates scores for combinations of feature patterns and instructor styles. The server analyzes feedback text with sentiment analysis or clustering algorithms to detect common issues or requests.

The server uses this feedback analysis to update the generative AI model and the prompt generation process. For example, the server may learn that users with low exercise time prefer encouraging tone rather than strict tone, and may adjust future prompt sentences by emphasizing positive reinforcement language when the feature vector indicates low activity. The server may also re-weight training examples during fine-tuning of the generative AI model so that outputs similar to positively rated messages are favored.

By integrating feedback into the training loop at the level of both prompt generation and model weights, the server alters the behavior of the computing system in a way that improves the quality and personalization of generated outputs. This process is not a mere automation of human selection; it exploits the computational ability of the server to discover non-obvious relationships between rich feature vectors and effective coaching language patterns.

### 7. Technical Effects and Improvements

The server improves computer functionality in several ways. First, the server uses structured feature transformation and dimensionality reduction to reduce the size and redundancy of input representations for the generative AI model, which decreases memory usage and speeds up inference. Second, the server uses rule-based and importance-weighted prompt construction that is specifically optimized for transformer-based generation, which stabilizes output quality and reduces the need for multiple retries, thus lowering compute and communication load.

Third, by integrating feedback-aware fine-tuning, the server modifies the generative AI model to concentrate its capacity on high-utility regions of the output space. This leads to improved generation accuracy as measured by user scores, and reduces the number of corrective interactions or manual interventions required. Fourth, the server's architecture for storing numerical feature vectors, prompt sentences, and feedback in a tightly coupled manner enables advanced analytics and automated adaptation that are not easily achievable with conventional static rule systems.

The system also yields technical improvements in data management. The server maintains a consistent data schema for user daily-life information, feature information, and behavior history, and performs automated transformations that are repeatable and verifiable. This reduces errors from ad hoc data handling and simplifies scaling to large numbers of users. Furthermore, the feature-based and prompt-based architecture allows the server to batch-process multiple users at once, performing vectorized computations on a hardware accelerator, thereby improving throughput.

By implementing these computational structures and methods, the system achieves tangible performance benefits such as faster response times for generating virtual instructors and advice messages, more precise matching of advice style to user profiles, and reduced bandwidth usage due to less need for repetitive communication in order to correct unsuitable outputs. These improvements are rooted in specific algorithmic designs, data structures, and model training techniques, rather than a mere business rule or human decision automation.

### 8. Variations and Alternative Embodiments

The server can employ various types of generative AI models. In an alternative embodiment, the server uses a sequence-to-sequence architecture with an encoder that processes the prompt sentence and a decoder that generates the profile information and advice messages. In another embodiment, the server uses a hybrid architecture in which a classifier predicts an instructor style category from the numerical feature vector, and a style-specific generative model generates advice messages conditioned on that category.

The feature transformation pipeline can also vary. In one embodiment, the server uses embedding layers trained jointly with the generative model to map categorical features directly into a continuous space. In another embodiment, the server uses an autoencoder to learn a compressed representation of user behavior sequences, and the latent code of the autoencoder is concatenated with the numerical feature vector used for prompt construction. The prompt sentence format may be modified while maintaining the core functionality of summarizing user characteristics and constraining the generative AI model. For example, the server may include explicit instruction tokens such as “Instruction:” and “Output format:” to further structure the prompt. The server may also adjust the language of the prompt sentence according to locale, and the same feature vector can yield prompt sentences in different languages while using a multilingual generative AI model.

The visualization on the terminal may also vary. The terminal may display a two-dimensional avatar, a three-dimensional character, or a simplified icon. The mapping from profile information to visual appearance can be implemented by a rule-based system or by a learned mapping. In each case, the technical principle remains that the terminal uses structured profile information from the server to instantiate a visual display object that is consistent with the generated instructor profile.

Through these embodiments and variations, the server, terminal, and user interact in a manner that allows the invention to be practiced in different environments and configurations, while preserving the technical features of structured feature transformation, prompt-based generative processing, and feedback-driven model and prompt updating.

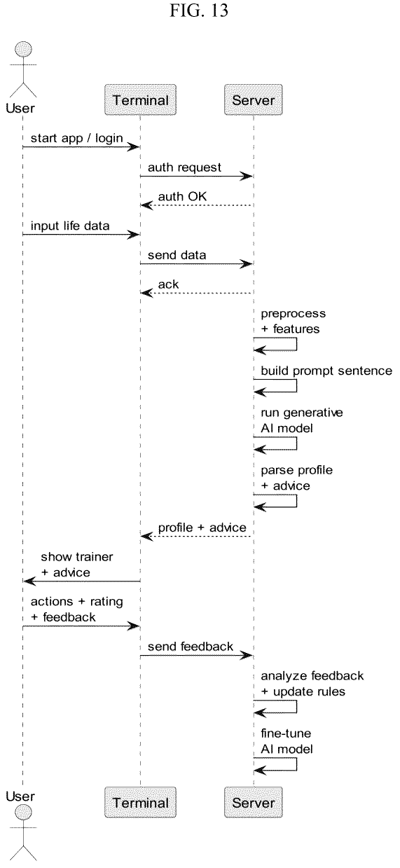

The following describes the processing flow using FIG. 13.

### Step 1:

User operates the terminal to start an application and authenticate.

User launches the application on the terminal and inputs authentication information such as an identifier and a password or approves a single sign-on dialog.

Terminal takes user authentication information as input, constructs an authentication request, and sends it to the server via a secure communication protocol.

Server receives the authentication request as input, compares the authentication information with registration data stored in a database, and outputs an authentication result including a session token when the information matches.

Terminal stores the session token locally and outputs an authenticated state that will be used as input for subsequent communication with the server.

### Step 2:

User operates the terminal to input daily-life information.

User views question screens on the terminal and inputs personality attributes, preferences, exercise logs, diet logs, goals, and optionally sensor-based activity data.

Terminal takes user-entered values and sensor readings as input and performs local validation such as type checking and range checking.

Terminal converts the validated information into an internal structured format, outputs a user daily-life information object containing fields such as personality type, exercise duration, meal descriptions, and timestamps, and stores it temporarily in local storage.

### Step 3:

Terminal transmits user daily-life information to the server.

Terminal takes the user daily-life information object and the session token as input, serializes the information into a structured message format, and attaches the token in a header field.

Terminal sends the message to the server through a communication interface using a network protocol.

Server receives the message as input, verifies the session token to identify the user, and parses the serialized content into a server-side data structure.

Server outputs a raw user data record and stores it in a persistent storage device associated with the user identifier.

### Step 4:

Server preprocesses user daily-life information into structured feature information.

Server takes the raw user data record as input and loads it into a tabular data processing environment that represents the data as rows and columns.

Server performs data cleaning operations such as removing invalid entries, filling missing values according to predetermined rules, normalizing time units, and unifying categorical labels.

Server aggregates multiple records by time windows, for example computing weekly exercise minutes and average daily energy intake, by summing or averaging appropriate columns.

Server outputs feature information that represents the user's personality, preference, lifestyle, exercise status, and dietary status in a structured form suitable for further numerical processing.

### Step 5:

Server converts feature information into a numerical feature vector.

Server takes the feature information as input and applies a feature transformation pipeline that includes encoding categorical fields, scaling numerical fields, and optionally transforming text-based fields.

Server executes encoding algorithms that map each categorical value to one or more numeric dimensions, and executes scaling algorithms that transform raw numeric values to normalized ranges such as zero mean and unit variance.

Server optionally applies dimensionality reduction to the transformed data to remove redundancy and reduce vector size.

Server combines all transformed components into a single ordered array and outputs a numerical feature vector associated with the user.

### Step 6:

Server generates a prompt sentence based on the numerical feature vector.

Server takes the numerical feature vector and corresponding labels such as exercise level and diet type as input and interprets numeric ranges using predefined rules to derive linguistic descriptors such as “high activity” or “moderate calorie intake.”

Server arranges these descriptors in a predetermined order of importance and inserts them into a template that defines sections such as “User personality,” “Exercise,” and “Diet.”

Server concatenates the sections into a continuous text string and adds explicit instructions for required output format and style of the virtual instructor.

Server outputs a prompt sentence in natural language that summarizes the user's characteristics and provides constraints for the generative AI model.

### Step 7:

Server executes a generative AI model with the prompt sentence to generate virtual instructor information.

Server takes the prompt sentence as input and converts the text to a sequence of tokens using a tokenizer associated with the generative AI model.

Server supplies the token sequence to a neural network that includes an embedding layer, multiple attention-based layers, and an output layer, and performs forward computation to obtain a probability distribution over output tokens at each position.

Server selects output tokens using a decoding strategy such as greedy selection or sampling with constraints on length and diversity, and converts the selected tokens back into text.

Server outputs generated text that includes a virtual instructor name, personality description, coaching style description, and multiple advice messages for exercise and diet.

### Step 8:

Server parses generated text into a structured virtual instructor profile and advice messages.

Server takes the generated text as input and analyzes it according to patterns or markers such as line prefixes and separators.

Server identifies segments corresponding to a name field, personality field, communication style field, and a list of advice entries, and checks whether each required field is present.

Server applies correction rules when necessary, such as discarding incomplete segments or regenerating missing parts using a refined prompt sentence.

Server outputs a virtual instructor profile object and an advice message list as structured data.

### Step 9:

Server transmits virtual instructor profile and advice messages to the terminal.

Server takes the virtual instructor profile object and the advice message list as input and serializes them into a structured response format.

Server attaches metadata such as identifiers and timestamps and sends the response to the terminal via the communication interface.

Terminal receives the response as input, deserializes the content into terminal-side data structures, and stores the information in local storage.

Terminal outputs ready-to-display virtual instructor data that includes both profile attributes and associated advice messages.

### Step 10:

Terminal generates and displays a visual representation of the virtual instructor with advice messages.

Terminal takes the virtual instructor profile as input and selects a visual asset or graphical template corresponding to the instructor's personality type or style.

Terminal composes a visual display object that includes an avatar image, the instructor name, and a short description, and arranges the advice messages below or next to this display object according to a layout rule.

Terminal renders the visual display object and advice messages on the display unit as a graphical user interface.

User views the virtual instructor and advice messages as output and can interact with the interface.

### Step 11:

User interacts with the virtual instructor and performs suggested activities.

User reads the advice messages displayed on the terminal and decides whether to perform suggested exercises or dietary actions.

User operates the terminal to indicate performed actions, for example by pressing completion buttons, selecting checkboxes, or entering actual exercise and diet records.

Terminal takes user interaction signals as input and updates local logs representing task completion information and behavior history information.

Terminal outputs updated status and may present progress indicators or additional messages to the user.

### Step 12:

Terminal collects evaluation information and feedback information from the user.

User operates evaluation controls on the terminal, such as rating scales or feedback text fields, to express satisfaction level and qualitative comments regarding the virtual instructor and the advice messages.

Terminal takes rating values and text entries as input and associates them with identifiers of the relevant virtual instructor instance, advice messages, and session.

Terminal constructs a feedback payload containing evaluation scores, feedback text, and context such as timestamps and user identifiers.

Terminal outputs the feedback payload and prepares it for transmission to the server.

### Step 13:

Terminal transmits evaluation information and feedback information to the server.

Terminal takes the feedback payload and the session token as input and sends them to the server using a secure communication protocol.

Server receives the feedback payload as input and validates its structure and associated session information.

Server writes the feedback information into a storage device, linking it to the corresponding numerical feature vector, prompt sentence, and generated outputs for later analysis.

Server outputs stored feedback records ready for batch processing and model updating.

### Step 14:

Server analyzes accumulated feedback and updates feature and prompt generation rules.

Server takes multiple stored feedback records, associated numerical feature vectors, and prompt sentences as input and loads them into a data processing environment.

Server computes aggregated metrics such as average satisfaction scores per feature pattern and detects correlations between user characteristics and preferred instructor styles or advice types using statistical methods.

Server modifies thresholds, weightings, or selection rules used in the feature-to-text mapping stage so that future prompt sentences emphasize patterns correlated with higher satisfaction.

Server outputs updated configuration parameters for feature transformation and prompt sentence generation.

### Step 15:

Server fine-tunes the generative AI model based on feedback.

Server takes training examples constructed from prompt sentences, generated outputs, and corresponding evaluation information as input and forms a dataset where each example includes a prompt and an output with an associated weight derived from user scores.

Server performs additional training passes on the generative AI model, computing a loss function that incorporates these weights so that positively rated examples contribute more strongly to parameter updates.

Server updates internal model parameters through gradient-based optimization, thereby adjusting token prediction behavior to favor patterns aligned with user preferences.

Server outputs an updated generative AI model that is better adapted to the distribution of real-world user feedback and integrates it into the inference subsystem for subsequent requests.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional health-management and coaching systems generally treat sensor data, dietary data, and user sentiment data as separate streams and process them using fixed, template-based logic. In such systems, the server typically aggregates step counts, calorie estimates, or basic questionnaire responses and then selects a pre-written message from a small library of generic advice. This architecture suffers from several technical limitations. First, existing systems lack a unified, time-sequenced representation that tightly correlates activity indices, nutrition indices, and emotional states. Data are often stored in siloed formats or separate services, which increases processing overhead for later analyses, complicates indexing and querying, and leads to latency and inconsistency when generating responses. As a result, the server cannot efficiently evaluate the user's state in context, and cannot dynamically adapt guidance in real time.

Second, emotion recognition is often implemented as a peripheral feature that only reads superficial sentiment labels and does not feed those labels back into core data structures or subsequent computation paths. Conventional architectures therefore cannot technically optimize their internal models or message-generation components using fine-grained emotional context. This yields low-relevance feedback and requires repeated, redundant computations to approximate personalization.

Third, known systems that integrate generative AI models commonly pass raw metrics or loosely structured text directly to such models. This ad-hoc prompting leads to unstable quality, unnecessary token usage, and increased network and compute overhead. There is no standardized, machine-generated prompt sentence that consistently encodes the server's computational results—such as goal-achievement status, fatigue level, and inferred preferences—into a compact natural-language instruction optimized for the generative model. This inefficiency directly degrades throughput on the server side and wastes computational resources in the generative AI infrastructure.

Fourth, many architectures treat a virtual trainer or coach as a static user-interface object. They do not maintain a structured instructor profile that includes personality characteristics, appearance characteristics, response style, and mapping rules from internal indices to external behaviors. As a consequence, the server must repeatedly re-compute the same presentation logic or hard-code behavior in the client, leading to poor scalability, limited adaptability, and increased maintenance cost.

Fifth, existing systems make limited use of transaction-related information, such as purchase history or location history, in the computational pipeline. In most cases such information is either ignored or only used in offline analytics, not in real-time message generation. This prevents the server from accurately estimating nutrient intake from meal candidates inferred from images and transaction logs in a computationally efficient, automated manner. Sixth, feedback from the user regarding the usefulness of previous messages and the user's subsequent behavior is rarely fed back into the server's prompt-generation logic and instructor profile management as a first-class, machine-readable signal. This results in a technically suboptimal loop, in which the system cannot systematically adapt its internal parameters, cannot optimize prompt sentences for future interactions, and cannot reduce unnecessary processing for patterns known to be ineffective for a specific user. Accordingly, there is a need for an improved computer-implemented system and server architecture that (i) constructs a unified, time-sequenced internal representation combining activity indices, nutrition indices, and emotional states; (ii) generates and maintains a structured instructor profile for a virtual instructor entity; (iii) automatically constructs optimized prompt sentences tailored for a generative AI model, based on internal computational results; (iv) integrates transaction-related information and image-based meal candidates to compute nutrition indices; and (v) adaptively modifies both prompt sentences and instructor profiles in response to user feedback and newly acquired daily-life information. Such improvements allow the server to reduce redundant computation, improve the relevance and consistency of generated messages, and enhance the overall efficiency of the computer system implementing personalized health guidance and emotional support.

The specific processing by the specific processing unit 290 of the data processing device 12 in Application Example 2 is realized by the following means.

The present invention provides a server comprising a processor configured to acquire information relating to a user's daily life from measurement apparatuses and information-processing applications; integrate the acquired information in a time sequence to generate activity indices and nutrition indices; analyze voice information and character information of the user by using a natural language processing algorithm and a voice-feature analysis algorithm; estimate an emotional state and associate the emotional state with the time-sequenced activity indices and nutrition indices; evaluate, based on the activity indices, the nutrition indices, and the emotional state, at least one of goal-achievement status, fatigue level, and preferences of the user; generate or update an instructor profile indicating personality characteristics, appearance characteristics, and response style of a virtual instructor entity; construct, on the basis of the activity indices, the nutrition indices, the emotional state, and the instructor profile, a prompt sentence written in a natural language and describing a current situation of the user, a desired guidance policy, and constraint conditions for a message; input the prompt sentence into a generative information generation model and obtain, from the generative information generation model, response information including at least one of an advice message and an encouragement message to be presented by the virtual instructor entity to the user; determine, on the basis of the response information, at least one of a display operation, an audio output, and dialog content of the virtual instructor entity and present the virtual instructor entity and at least one of the advice message and the encouragement message on a portable information-processing apparatus or a display apparatus; acquire transaction-related information including at least one of behavior history, purchase history, and location history of the user from a recording medium; integrate meal-candidate information obtained by image analysis with the transaction-related information to estimate intake amounts of nutrients and incorporate an estimation result into the prompt sentence; and adaptively modify, based on feedback information from the user and additional daily-life information acquired after presentation of at least one of the advice message and the encouragement message, contents of a subsequent prompt sentence and the instructor profile to be generated thereafter. This enables the computer system to internally maintain a unified, time-sequenced model of activity, nutrition, and emotional data; to algorithmically derive a structured instructor profile; to automatically construct optimized prompt sentences as machine-generated instructions for the generative AI model; to compute more accurate nutrition indices by combining image-based meal candidates with transaction-related information; and to continuously refine both prompts and instructor behavior based on user feedback, thereby improving computational efficiency, stability and personalization of AI-generated guidance compared to conventional systems.

The term “information relating to a user's daily life” refers to data describing activities, behaviors, and conditions of the user in everyday situations, including at least physical activity, dietary intake, and contextual information such as time and location.

The term “measurement apparatus” refers to an electronic device configured to sense or measure physical quantities related to the user, such as motion, heart rate, or location, and to output the measured values as digital data.

The term “information-processing application” refers to a software program executed on an electronic device, the software program being configured to receive user input, access device functions, and generate or transmit digital data describing the user's daily life.

The term “activity index” refers to a numerical or categorical value derived from raw sensor data that quantifies a characteristic of the user's physical activity over a defined time period, such as step count, distance moved, active duration, or exercise intensity.

The term “nutrition index” refers to a numerical or categorical value derived from meal-related information that quantifies a characteristic of the user's dietary intake over a defined time period, such as total energy intake, macronutrient balance, or estimated intake of specific nutrients.

The term “voice information” refers to audio data representing speech uttered by the user, including waveform signals and any features or transcriptions derived from the audio data.

The term “character information” refers to text data associated with the user, including user-entered text, transcribed speech, and text obtained from external sources such as messages or posts.

The term “natural language processing algorithm” refers to a computational procedure executed by the processor to analyze, interpret, or generate human language text, including at least tokenization, syntactic analysis, semantic analysis, and sentiment or emotion classification.

The term “voice-feature analysis algorithm” refers to a computational procedure executed by the processor to extract and analyze acoustic features from voice information, such as pitch, energy, tempo, or spectral characteristics, for purposes including emotion estimation or speaker state detection.

The term “emotional state” refers to a condition of the user's affect or mood inferred from voice information, character information, or other signals, and represented as at least one label, score, or category such as joy, sadness, fatigue, or motivation level.

The term “instructor profile” refers to structured data describing configuration parameters of a virtual instructor entity, including at least personality characteristics, appearance characteristics, and response style to be applied when generating or presenting content to the user.

The term “personality characteristic” refers to an attribute within the instructor profile that defines a behavioral tendency or communication style of the virtual instructor entity, such as being energetic, calm, strict, or empathetic.

The term “appearance characteristic” refers to an attribute within the instructor profile that defines a visual or auditory aspect of the virtual instructor entity, such as avatar style, facial expression baseline, clothing, or voice tone.

The term “response style” refers to a set of rules or parameters in the instructor profile that governs how the virtual instructor entity formulates and presents messages, including tone, level of detail, directness, and frequency of encouragement or warnings.

The term “virtual instructor entity” refers to a computer-generated representation, including at least one of an avatar, an agent, or a character, configured to present advice or encouragement to the user via a user interface.

The term “prompt sentence” refers to a text sequence expressed in a natural language, constructed by the processor, and used as an input instruction to a generative information generation model to control content, style, or constraints of a generated output.

The term “generative information generation model” refers to a trained computational model that receives a prompt sentence and optionally additional context as input and outputs newly generated information, such as text forming an advice message or an encouragement message.

The term “response information” refers to data output from the generative information generation model in response to a prompt sentence, the data including at least one of an advice message and an encouragement message intended for presentation to the user.

The term “advice message” refers to text or audio content generated for the purpose of guiding the user's behavior, such as recommending specific physical activities, dietary changes, or rest strategies.

The term “encouragement message” refers to text or audio content generated for the purpose of motivating or emotionally supporting the user, such as praising achievements or providing reassurance.

The term “display operation” refers to a control instruction or sequence that defines how the virtual instructor entity or associated content is visually rendered on a display apparatus, including animations, layout, and timing of presentation.

The term “audio output” refers to sound reproduced by a speaker or audio device under control of the processor, including at least synthesized speech corresponding to messages of the virtual instructor entity.

The term “dialog content” refers to a sequence of messages, questions, and responses exchanged between the virtual instructor entity and the user, where the messages are determined based on response information and internal state.

The term “portable information-processing apparatus” refers to a handheld or wearable electronic device configured to execute an information-processing application and to communicate with the server, such as a mobile terminal, a tablet terminal, or a wearable terminal.

The term “display apparatus” refers to an electronic device having at least one display unit configured to visually present images, text, or avatars controlled by the processor.

The term “transaction-related information” refers to data describing at least one of user behavior history, purchase history, or location history, obtained from a storage medium or an external service, and usable to infer aspects of the user's lifestyle or dietary intake.

The term “behavior history” refers to recorded information about user actions over time, including at least visits to facilities, movement patterns, or use of services.

The term “purchase history” refers to recorded information about goods or services acquired by the user, including item identifiers, categories, timestamps, and amounts.

The term “location history” refers to recorded information about geographical positions associated with the user over time, including coordinates, regions, or place identifiers.

The term “recording medium” refers to any physical or logical storage resource capable of storing digital data, including at least a local memory, a storage device, or a remote storage service.

The term “meal-candidate information” refers to data representing possible food items or dishes associated with an eating event, obtained from analysis of image data or other contextual data.

The term “image analysis” refers to a computational process that examines digital image data to detect, classify, or recognize objects or patterns, including at least food items in a meal image.

The term “intake amount of nutrients” refers to an estimated quantity of one or more nutritional components, such as energy, protein, fat, carbohydrates, vitamins, or minerals, consumed by the user during a defined time period.

The term “feedback information” refers to data indicating user reactions or evaluations to previously presented messages or behaviors of the virtual instructor entity, including explicit inputs such as ratings, selections, or comments, and implicit signals such as subsequent user behavior.

The term “additional daily-life information” refers to information relating to the user's daily life that is acquired after presentation of an advice message or an encouragement message, including new activity data, new dietary data, or new emotional data.

The term “subsequent prompt sentence” refers to a prompt sentence generated after at least one advice message or encouragement message has been presented, the prompt sentence being constructed in consideration of prior interactions and newly acquired data.

The term “time-sequenced” refers to a state in which data elements are organized according to their occurrence times, enabling chronological indexing, retrieval, and analysis.

The term “goal-achievement status” refers to a result of evaluating whether a predefined target relating to activity or diet has been met, partially met, or not met within a specific period.

The term “fatigue level” refers to an estimated degree of physical or mental tiredness of the user, derived from at least activity indices, emotional state, or user-provided information.

The term “preference” refers to an inferred or declared inclination of the user regarding content, style, or intensity of guidance, including preferred tone of messages, frequency of notifications, or type of virtual instructor persona.

The term “summary sentence” refers to a text sequence in a natural language that concisely describes numerical or categorical information, including the activity indices, the nutrition indices, and the emotional state, in a human-readable form.

The term “instruction sentence optimized for the generative information generation model” refers to a prompt sentence whose structure, wording, and included context are selected or adjusted to improve the quality, relevance, or stability of the output produced by the generative information generation model.

In one embodiment, a server, a plurality of terminals, and at least one user cooperate to implement the claimed system. The server includes at least one processor, a memory storing executable programs and data structures, and one or more network interfaces. The terminal includes a processor, a memory, a display unit, at least one sensor (such as an accelerometer, a heart-rate sensor, or a positioning sensor), an audio input unit, and a communication interface. The user carries or wears the terminal and interacts with the server through an application.

The server executes an operating system and server-side application software implemented, for example, using a general-purpose programming language such as Python or a web framework such as a typical server framework. The server stores user data in a database system such as a document-oriented data store or a relational database system. The server stores learned parameters of neural network models in the memory or the database and loads them into main memory as needed.

The terminal executes an information-processing application installed on a mobile operating system. The application accesses sensors of the terminal and of connected wearable devices, such as a smartwatch or a fitness tracker, via standard sensor APIs. The terminal acquires acceleration signals, heart-rate values, and positioning coordinates at predefined sampling intervals. The terminal also acquires images from a camera, voice data from a microphone, and character information from user input forms.

The server defines a specific data structure to represent information relating to a user's daily life. In one example, the server uses a time-series record structure in which each record includes a user identifier, a timestamp, a data type (activity, meal, emotion, transaction), and a payload field. The payload includes normalized values such as step count per minute, distance traveled, calculated calories, recognized food items, estimated nutrient values, emotion labels, and transaction identifiers. The server organizes these records in chronological order and maintains indexes on the user identifier and timestamp to support efficient range queries.

The server aggregates raw sensor values into activity indices. The server receives acceleration vectors and heart-rate samples from the terminal and executes, on the processor, an activity-detection algorithm implemented using numerical libraries. The server segments the time-series into fixed-length windows, computes features such as mean acceleration magnitude, variance, step frequency, and average heart rate, and classifies each window into activity categories such as walking, running, or resting using a trained classifier model. The server then sums the classified segments to generate daily and weekly activity indices including total steps, total distance, active time, and intensity scores.

The server aggregates meal-related data into nutrition indices. The server receives image files captured by the terminal at mealtimes and uses image analysis software to detect meal-candidate information. In one example, the server employs a convolutional neural network including multiple convolutional layers, pooling layers, and fully connected layers to classify food items present in the image. The server maps the predicted food classes to a food dictionary that contains nutrient values per unit portion. The server estimates the portion size using bounding-box areas and calibration data and computes an estimated intake amount of nutrients such as energy, protein, fat, and carbohydrates.

The server further supplements nutrition indices with transaction-related information. The server acquires purchase history and location history from a recording medium or an external service. The server links transaction records to meal events by matching timestamps and location coordinates. The server uses a rule-based parser to map product descriptions in the purchase history to known food items. The server integrates this transaction-based meal-candidate information with the image-based meal-candidate information using a weighted fusion rule. For example, the server increases the confidence of a food item when both image analysis and transaction parsing indicate the same item, and the server discards low-confidence items that conflict with transaction records. This integration improves the precision of the estimated intake amounts of nutrients and reduces errors caused by image misclassification.

The server estimates an emotional state of the user using voice information and character information. The server receives character information such as diary text, chat messages, and transcribed voice data from the terminal. The server tokenizes the character information and converts tokens into embeddings using a word or subword embedding layer. The server inputs the embeddings into a neural network based on a sequence model architecture, such as a bidirectional recurrent network or a transformer-type encoder, trained to output probability distributions over emotion categories such as joy, sadness, fatigue, stress, and motivation. The server chooses the emotion category with the highest probability and stores it as the emotional state in association with the corresponding time-sequenced activity indices and nutrition indices.

The server analyzes voice information by extracting acoustic features such as pitch, energy, speaking rate, and spectral coefficients. The server applies a feature normalization process and feeds the features into a classifier network dedicated to paralinguistic emotion recognition. The server fuses the emotion estimates from character information and voice information using a rule such as weighted averaging of probabilities or a learned fusion layer. This fusion allows the server to increase robustness of emotion recognition under noisy conditions.

The server maintains an instructor profile as a structured object stored in the database. The instructor profile includes fields indicating personality characteristics, appearance characteristics, and response style for a virtual instructor entity. For example, personality characteristics include numerical scores for dimensions such as cheerfulness, strictness, empathy, and talkativeness. Appearance characteristics include avatar style identifiers, color scheme parameters, and facial expression baselines. Response style includes parameters controlling message length, frequency of encouragement, level of detail in explanations, and preference for direct commands versus suggestions.

The server updates the instructor profile based on activity indices, nutrition indices, emotional state, and user feedback information. The server uses a mapping model, for example a feed-forward neural network with fully connected layers, to transform a vector representation of recent user state and past feedback outcomes into updated instructor-profile parameters. The server stores the updated instructor profile and uses it as a reference when generating subsequent messages and presentation behaviors of the virtual instructor entity. The server constructs a prompt sentence in natural language to control a generative AI model. The server first generates a summary sentence from numerical and categorical data. The server maps activity indices, nutrition indices, and emotional state into a compact textual description such as “Today the user walked 8,500 steps, ran 4 kilometers, and consumed approximately 2,100 kilocalories with slightly low protein intake, and the user expressed feeling tired.” The server then appends information derived from the instructor profile, such as “The virtual instructor is an energetic but gentle running coach who prefers concise messages.” The server finally appends explicit instructions to the generative AI model regarding desired output type, tone, and constraints, forming the prompt sentence. In one concrete example, the server constructs the following prompt sentence: “Given the following context: Today the user walked 8,500 steps, ran 4 km, and consumed approximately 2,100 kcal with slightly low protein. The user wrote ‘I get tired easily these days.’ The virtual instructor is an energetic but gentle running coach. Generate a short, encouraging message that acknowledges the user's fatigue, praises today's 4 km run, and proposes a slightly lighter training plan for tomorrow, including a reminder about protein intake. The message should be friendly, concise, and in English.”

In another concrete example, the server constructs a prompt sentence for a user who has achieved a goal but has low mood:

“The user ran 5 km today but wrote ‘I feel exhausted and a bit discouraged.’ You are a calm and supportive virtual trainer. Generate a short, empathetic message that acknowledges the effort, validates the exhaustion, and gently encourages rest and a lighter workout tomorrow, while reminding the user that such days are normal.”

In yet another example, the server constructs a prompt sentence aimed at nutrition advice: “The user's meals today totaled approximately 1,600 kcal with low protein and high carbohydrates. The user also posted ‘I get tired easily these days.’ As a friendly nutrition coach, generate a concise message suggesting specific high-protein foods the user can add to tomorrow's meals to improve energy levels.”

The server inputs the constructed prompt sentence into a generative AI model. The generative AI model is, for example, a transformer-based language model comprising multiple self-attention layers and feed-forward layers, with learned parameters stored in the memory. The server communicates with the generative AI model either as a local service or as a remote service over a network interface. The server transmits the prompt sentence as textual input and receives response information comprising an advice message and/or an encouragement message.

The server post-processes the response information. The server checks whether the length of the generated text is within predefined limits, removes inappropriate content according to safety rules, and can insert user-specific placeholders such as the user's name. The server classifies the response information into message types such as “motivation,” “training plan,” or “nutrition tip” using a lightweight classifier model or rule-based heuristics. The server stores the final message together with the prompt sentence in the database for later analysis. The server determines display operations, audio outputs, and dialog content of the virtual instructor entity based on the response information and the instructor profile. The server generates a control sequence specifying avatar animation identifiers, facial expression changes, and timing for message display. The server also generates text-to-speech parameters such as speaking rate and pitch to match the personality characteristics of the instructor profile. The server transmits the text message and control sequence to the terminal over a network.

The terminal presents the virtual instructor entity and the generated messages on a display apparatus. The terminal runs a rendering component, for example a graphics engine or a game engine, and uses avatar models pre-stored in local memory. The terminal applies the appearance characteristics and animation identifiers received from the server to render the avatar on the display. The terminal displays the advice message or encouragement message as text, and optionally converts the text to audio using a text-to-speech engine, outputting the synthesized speech through a speaker. The user thus perceives the guidance and encouragement as a combination of visual and auditory stimuli in real time.

The user responds to the guidance by performing physical activities, adjusting meals, or resting, and may also provide explicit feedback through the terminal. The user can input evaluations such as “This advice was helpful” or “This was too strict,” and can answer simple questions presented by the virtual instructor entity. The user's subsequent behavior, as captured by the sensors and transaction history, serves as implicit feedback.

The server collects feedback information and additional daily-life information acquired after presentation of the message. The server labels interactions with outcome values such as “user followed advice” or “user did not change behavior” by analyzing sensor-derived activity indices and nutrition indices after message delivery. The server uses these outcome values as targets in a learning process or as signals in an adaptation algorithm for updating the instructor profile and for changing the rules by which prompt sentences are constructed. For example, the server decreases a strictness parameter in the instructor profile when repeated strict messages correlate with low adherence.

The server implements specific internal modules to improve computer-technical performance. The server includes a prompt-construction module that caches partial natural-language templates for repeated patterns of activity and nutrition indices, thereby reducing computation time for template generation. The server includes an emotion-fusion module that uses vectorized representations and efficient matrix operations to combine multiple emotion signals. The server also includes a nutrition-estimation module that uses indexed lookup tables and precomputed nutrient vectors to quickly sum nutrient intake from identified food items. These modules reduce CPU load and response latency compared with naive string concatenation and ad-hoc computations for each request.

The server improves communication efficiency by minimizing data transmitted to the generative AI model. By converting high-dimensional numeric features into a concise prompt sentence, the server reduces the number of tokens sent to and processed by the generative AI model. This reduction lowers network traffic and computational cost on the generative AI infrastructure, thereby enabling faster response times and reduced resource consumption. The server further improves communication efficiency between the server and the terminal by sending high-level control directives (for example, animation identifiers and style parameters) instead of raw frame sequences, enabling the terminal to render rich interactions with low bandwidth.

The server improves accuracy and stability of AI-generated guidance by implementing a two-stage prompt-generation procedure. In one variation, the server first generates a summary sentence purely from numeric and categorical data, and then generates a final prompt sentence by optimizing the wording and structure of the instruction to the generative AI model. This separation allows the server to test or update the intermediate summary logic independently of the generative model. The server can adjust summary granularity and instruction wording based on offline evaluations of output quality, thus systematically improving the effectiveness of prompts rather than relying on ad-hoc human-written instructions.

The server improves data management by maintaining a unified, time-sequenced internal representation. By indexing all activity indices, nutrition indices, emotional states, and feedback information on a common time axis and user identifier, the server can perform complex queries and join operations with low overhead. This arrangement avoids duplication of data across multiple, inconsistent stores and reduces the need for repeated joins at query time. As a result, the server can create prompt sentences and updated instructor profiles quickly even when historical data volumes are large.

The server trains the various neural networks using pre-collected datasets and then deploys the trained models as part of the system. The server uses a training process in which it defines a loss function appropriate to each task: for activity classification, a cross-entropy loss over activity classes; for food recognition, a cross-entropy loss over food classes; for emotion classification, a cross-entropy loss over emotion categories; and for instructor-profile mapping, a regression or multi-task loss over profile parameters. The server updates model weights using an optimization algorithm such as stochastic gradient descent or an adaptive variant. The server can employ data augmentation methods such as adding noise to acoustic features, randomly cropping images, or paraphrasing texts to improve robustness.

The server uses non-conventional rules and processing flows that differ from simple human decision trees. For example, the server adapts the structure of the instructor profile and of the prompt sentences in real time based on feedback and performance outcomes, something that is difficult for a human coach to perform consistently and at scale. The server uses learned mappings from numeric indices and feedback outcomes to profile changes, rather than a fixed set of rules, thereby enabling the system to adjust itself to individual users in a high-dimensional parameter space. This leads to improved personalization and reduced manual tuning of templates.

Alternative embodiments are possible. In one alternative, the server executes the generative AI model locally rather than accessing a remote service, reducing network latency and allowing tighter integration with internal data structures. In another alternative, the terminal performs part of the emotion recognition, such as local speech-to-text and on-device sentiment classification, and sends only intermediate features to the server, further reducing network traffic. In yet another alternative, the server employs different neural network architectures such as convolutional sequence models or graph-based models to fuse temporal activity patterns with nutrition and emotion data.

In all these embodiments, the server, the terminal, and the user cooperate in a closed loop: the terminal acquires raw sensor data and user input; the server processes the data into activity indices, nutrition indices, and emotional states; the server generates prompt sentences and uses a generative AI model to generate guidance; the terminal presents the virtual instructor entity and messages; the user responds and provides feedback; and the server adapts its internal models and prompt-generation procedure. By designing specific data structures, model architectures, and processing flows as described above, the system achieves technical effects such as improved processing speed, improved accuracy of emotion and nutrition estimation, reduced communication load, and more stable and personalized AI-generated guidance than conventional, template-based or purely manual systems.

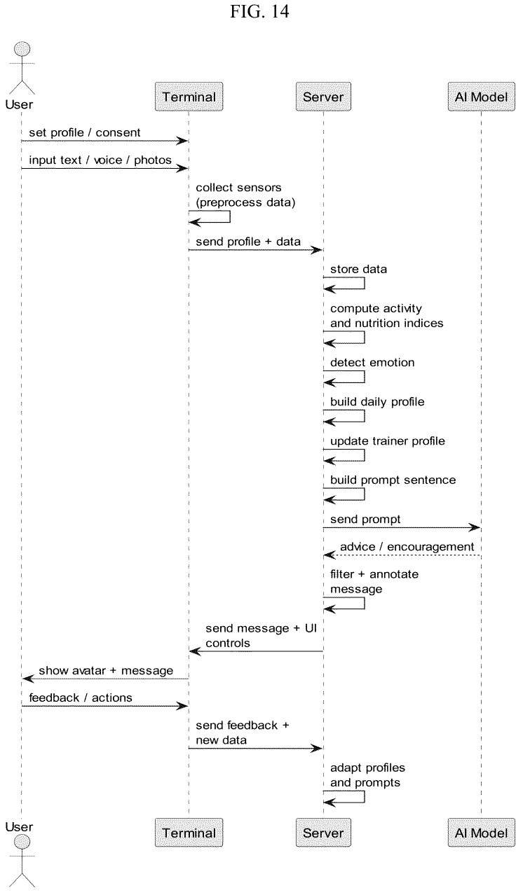

The following describes the processing flow using FIG. 14.

### Step 1:

User configures profile and consent settings in the terminal application.

User inputs age, height, weight, fitness goals, dietary preferences, and preferred trainer style into forms provided by the terminal.

Input: manual text and numeric entries from the user.

Terminal converts these entries into structured key-value pairs (for example, JSON fields) and validates ranges (for example, non-negative weight, realistic height).

Output: a validated profile object containing user attributes and preference parameters, stored locally and prepared for transmission to the server.

### Step 2:

Terminal acquires raw sensor data and contextual data.

Terminal reads acceleration, gyroscope, heart-rate, and positioning signals from embedded sensors and from connected wearable devices, and periodically samples values at predefined intervals. Terminal also captures images using the camera at user-indicated mealtimes and records voice data and text data via the microphone and text input fields.

Input: continuous sensor streams (acceleration vectors, heart-rate samples, GPS coordinates), camera images, microphone audio, and typed text.

Terminal timestamps each sample, converts raw hardware-specific formats into standardized numeric arrays, and groups them into short time windows.

Output: a collection of time-stamped raw data packets representing motion, heart activity, location, meal images, audio clips, and text logs.

### Step 3:

Terminal preprocesses raw data into structured daily-life records.

Terminal segments motion data into windows, computes simple features such as mean acceleration magnitude and step counts using peak-detection algorithms, and derives per-minute or per-hour movement summaries. Terminal associates each meal image with a timestamp and a tag such as breakfast, lunch, or dinner, and converts voice recordings into text using a speech-to-text engine available on the device or in a connected service.

Input: raw data packets from Step 2.

Terminal performs data aggregation (for example, summing steps per minute), noise filtering (for example, low-pass filtering acceleration), and metadata attachment (for example, user

ID, time, location).

Output: normalized daily-life records including activity summaries, labeled meal images, transcribed text, and associated metadata, ready for upload.

### Step 4:

Terminal transmits profile and daily-life records securely to the server.

Terminal opens a secure network connection and sends the profile object from Step 1 together with the structured records from Step 3 to a predefined server endpoint.

Input: validated profile object and normalized daily-life records.

Terminal packages the data into request payloads, adds authentication tokens, and compresses or batches records to reduce communication overhead.

Output: authenticated request messages containing user profile and daily-life data delivered to the server.

### Step 5:

Server receives, validates, and stores user data.

Server parses incoming payloads, verifies authentication tokens, and checks schema consistency of the profile and daily-life records.

Input: request messages from Step 4.

Server discards invalid fields, fills in missing optional values with defaults, and writes accepted records into time-indexed storage tables or documents keyed by user identifier.

Output: persisted profile data and daily-life records stored in a unified time-sequenced data structure in the server's database.

### Step 6:

Server computes activity indices from motion and heart-rate data.

Server loads time-stamped acceleration and heart-rate records from storage and groups them by user and by day.

Input: motion and heart-rate records from Step 5.

Server applies windowing to the time-series, calculates features such as step frequency, average heart rate, and movement variance, and applies a trained activity classifier to label segments as walking, running, or resting. Server then aggregates labeled segments to calculate daily and weekly totals (steps, distance, active minutes, intensity scores).

Output: activity indices representing physical activity levels over each time interval, stored as structured numerical fields.

### Step 7:

Server computes nutrition indices from meal images and transaction-related information.

Server retrieves meal images and linked transaction records, such as purchase history and location history.

Input: meal images and transaction-related records from Step 5.

Server runs an image-recognition model on each meal image to identify food items and coarse portion sizes, and parses transaction descriptions to identify purchased food items and quantities. Server fuses the results using a rule-based or learned weighting scheme and maps identified items to nutrient tables, summing energy and nutrient components per meal and per day.

Output: nutrition indices including estimated daily nutrient intake values and quality indicators (for example, protein sufficiency, carbohydrate ratio), stored in the database.

### Step 8:

Server estimates emotional state from character information and voice-derived features.

Server reads character information such as diary text and transcribed speech, and acquires acoustic features derived from recorded voice signals.

Input: text fields and acoustic feature vectors extracted from data stored in Step 5.

Server tokenizes text, encodes tokens into embeddings, and inputs them into a trained sequence classifier to obtain probabilities for emotion categories. Server combines text-based emotion probabilities with voice-based estimates by averaging or using a fusion layer, and selects the most probable emotion category.

Output: emotion labels and associated confidence scores, assigned to time intervals and written into emotion records.

### Step 9:

Server generates a unified daily profile by joining activity indices, nutrition indices, and emotional states.

Server aligns activity, nutrition, and emotion data on a common timeline and groups them by user and by day.

Input: activity indices from Step 6, nutrition indices from Step 7, emotion labels from Step 8.

Server performs temporal joins, interpolating or snapping values when necessary, and computes summary metrics such as total steps on high-fatigue days or average protein intake when the user reports being tired.

Output: unified daily profile objects containing time-aligned activity, nutrition, and emotion summaries for each user and date.

### Step 10:

Server updates the instructor profile based on the unified daily profile and past feedback.

Server reads the current instructor profile and historical feedback data for the user.

Input: unified daily profile from Step 9 and stored instructor-profile and feedback records.

Server transforms the profile and feedback into feature vectors, applies a mapping model to compute updated personality, appearance, and response-style parameters, and enforces bounds (for example, preventing extreme strictness values).

Output: a revised instructor profile reflecting the user's recent state and preferences, stored back into the profile repository.

### Step 11:

Server constructs a summary sentence describing the user's current state.

Server extracts key fields from the unified daily profile and the updated instructor profile, such as steps, distance, nutrient deviations, and emotion labels.

Input: unified daily profile and updated instructor profile from Step 10.

Server formats numeric values into natural-language phrases and concatenates them into a coherent sentence describing the main aspects of the day while respecting length thresholds.

Output: a summary sentence in natural language that encapsulates the user's current activity, nutrition, and emotional context.

### Step 12:

Server constructs a prompt sentence for the generative AI model.

Server takes the summary sentence and appends explicit instructions derived from the instructor profile regarding tone, length, and focus of the generated message.

Input: summary sentence from Step 11 and updated instructor profile from Step 10.

Server adds constraints such as required message type (for example, encouragement plus plan suggestion), preferred language, and specific content elements, thereby forming a complete prompt sentence.

Output: a prompt sentence expressed as a single text input suitable for the generative AI model.

### Step 13:

Server calls the generative AI model and obtains response information.

Server transmits the prompt sentence to a generative AI model via a model interface and sets parameters such as maximum output length and sampling temperature.

Input: prompt sentence from Step 12.

Server waits for the model to generate output text, checks for network or timeout errors, and receives one or more candidate messages.

Output: response information containing an advice message and/or an encouragement message in natural language.

### Step 14:

Server filters and annotates the generated message.

Server evaluates the generated text against policy rules, such as forbidden topics or maximum length, and may truncate or discard non-compliant segments.

Input: response information from Step 13.

Server adds metadata to the message, such as a message type label and a reference to the prompting context, and stores the final message in the message history for future analysis.

Output: a sanitized and annotated message object ready for presentation to the user.

### Step 15:

Server determines presentation parameters and sends control data to the terminal.

Server selects avatar animations, facial expressions, and speech parameters that match the instructor profile and the content of the message.

Input: annotated message object from Step 14 and updated instructor profile from Step 10.

Server generates a control structure containing the text message, animation identifiers, timing instructions, and optional speech synthesis settings, and transmits this structure to the terminal over the network.

Output: a presentation package including text and control directives delivered to the terminal.

### Step 16:

Terminal renders the virtual instructor entity and presents the message.

Terminal receives the presentation package and loads a corresponding avatar model and animation set.

Input: presentation package from Step 15.

Terminal displays the avatar on the screen, executes the specified animations in synchrony with the message text, and optionally converts the text to speech and plays the audio.

Output: a visual and auditory presentation of the virtual instructor entity and the guidance message to the user.

### Step 17:

User observes the presentation and provides explicit and implicit feedback.

User reads or listens to the message and may tap quick-response buttons (for example, “OK,” “Too hard,” “More details”) or enter free-form comments. User may also follow or ignore the advice, resulting in different subsequent activity and meal patterns.

Input: presented message and avatar behavior from Step 16.

User generates explicit signals through input interactions and implicit signals through subsequent real-world actions.

Output: explicit feedback entries and changed or unchanged behavior patterns, both of which can be sensed by the system.

### Step 18:

Terminal captures feedback information and new daily-life data.

Terminal records the user's button presses, typed comments, and new sensor data following the message presentation.

Input: user interactions and post-message sensor data.

Terminal tags feedback records with identifiers of the message and prompt used, aggregates new motion and meal data as in Step 3, and prepares both for transfer to the server.

Output: structured feedback records and additional daily-life records reflecting user reactions.

### Step 19:

Server updates models and future prompt-generation logic using feedback.

Server receives new feedback and daily-life data, associates them with the previous message and prompt, and infers adherence or non-adherence to the advice by comparing planned versus actual activity and nutrition indices.

Input: feedback records and additional daily-life records from Step 18.

Server adjusts instructor-profile parameters and modifies weighting rules and templates used in summary and prompt construction, for example reducing strictness if harsh messages consistently correlate with poor adherence.

Output: updated instructor profile and refined rules for constructing future summary sentences and prompt sentences, completing the adaptive loop for subsequent executions.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

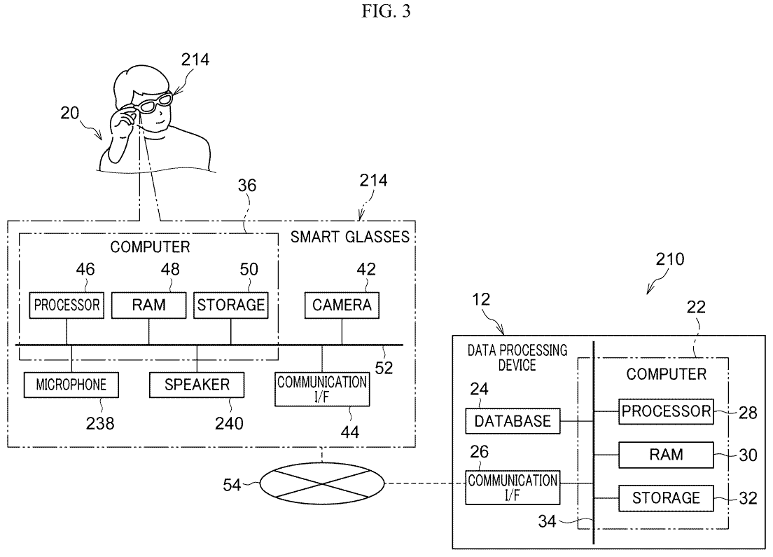

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

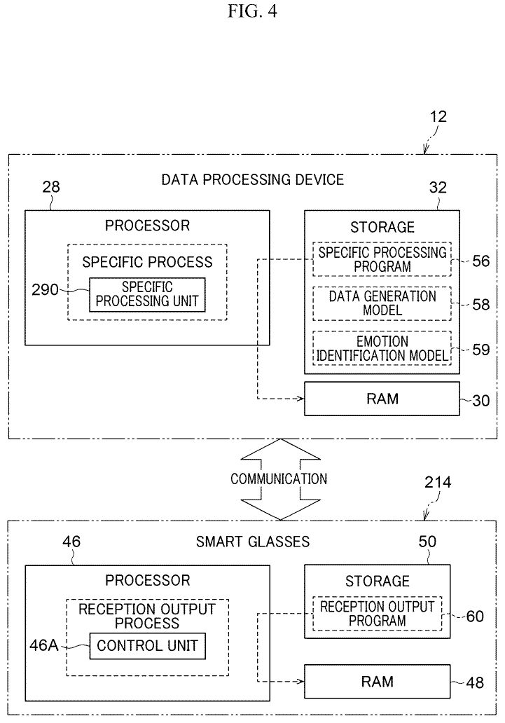

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

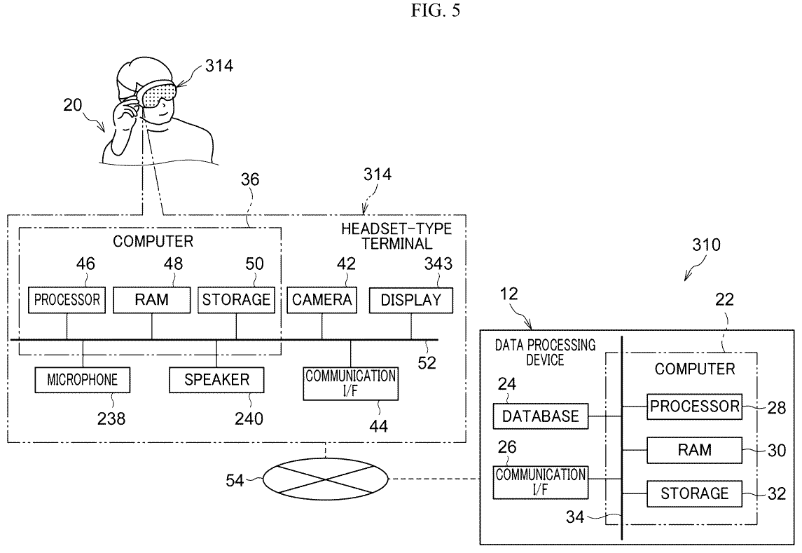

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

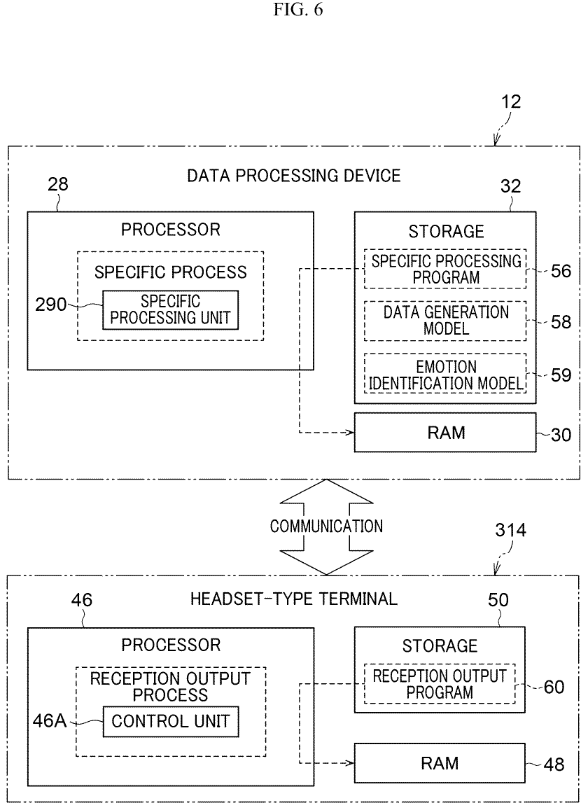

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

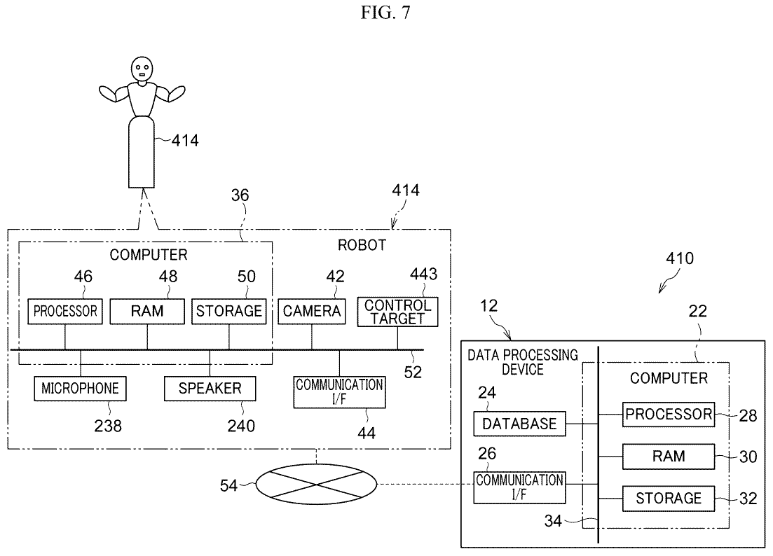

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

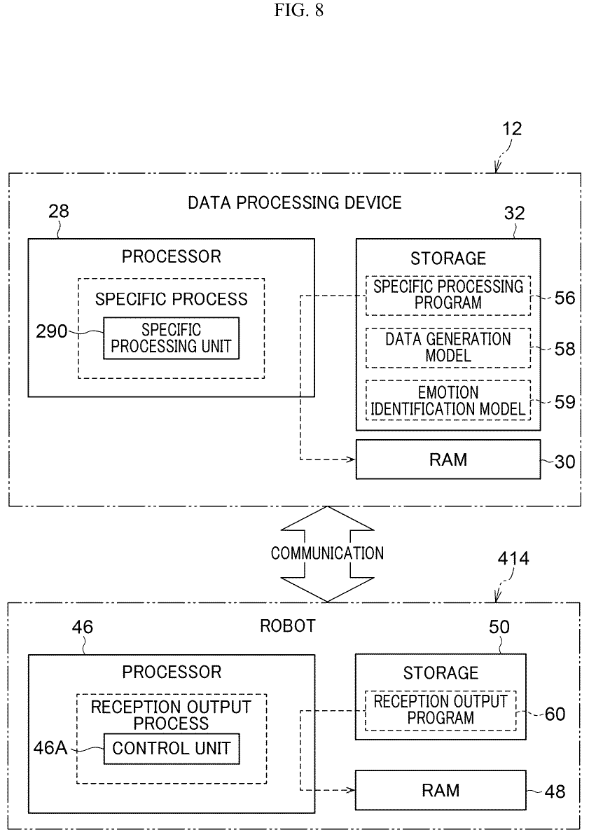

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

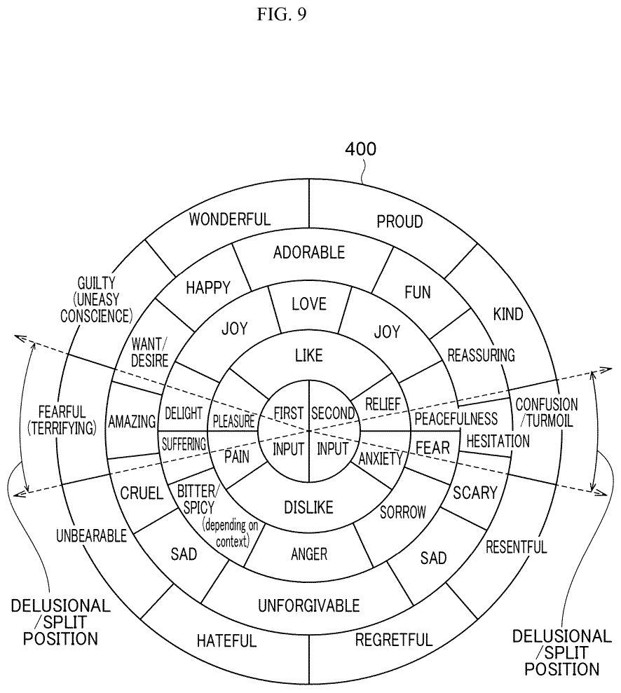

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

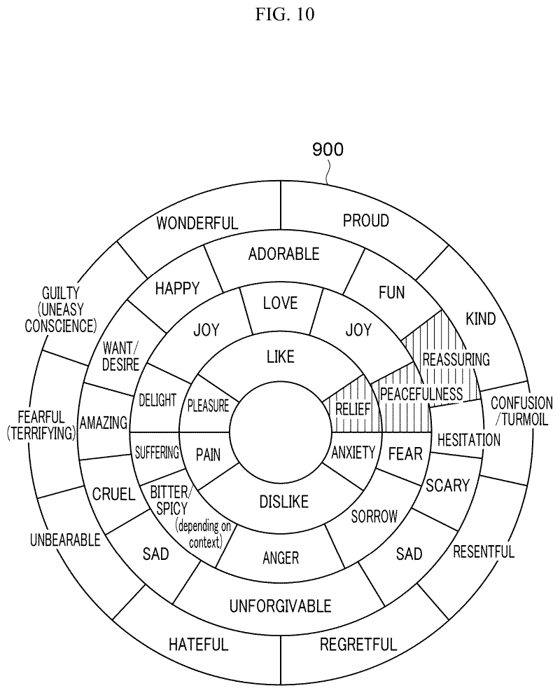

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire time-series data relating to a user's daily life, via information acquisition devices having a plurality of types of detection functions and via an information processing program, and integrate the time-series data for each user and for each period so as to store the time-series data as structured data including body activity data, location data, intake data, and transaction data,
analyze the structured data to calculate aggregated values of body activity, amounts of location movement, intake contents, and estimated nutrition values, classify the intake contents by using an image analysis algorithm and a collation process with the transaction data, and generate lifestyle indices and health state indices of the user,
generate a prompt sentence that specifies task contents and an output format, by using summary data of the user including the lifestyle indices and the health state indices as input information, and construct a model input sentence in which the prompt sentence incorporates the summary data,
transmit the model input sentence to a generative artificial intelligence model having a natural language generation function, receive a natural language evaluation result and advice contents from the generative artificial intelligence model, and analyze the natural language evaluation result and the advice contents to extract health evaluation information and lifestyle improvement proposal information,
generate additional prompt sentences including style information indicating attribute information of a virtual instructor, apply the additional prompt sentences and the advice contents to the generative artificial intelligence model, and thereby generate an advice text in a representation format of an instructor that imitates a human image or a character image according to a preference of the user, and
transmit the advice text to an information display terminal having a notification function and a display function and cause the information display terminal to present the advice text to the user.

### (Supplementary 2)

The system according to supplementary 1,
wherein the processor is configured to generate the structured data by automatically acquiring the body activity data and the location data at predetermined time intervals in cooperation with detection functions of a portable information terminal carried by the user, and to transmit acquisition results to a server device via encrypted communication so as to manage the acquisition results in real time or in quasi real time.

### (Supplementary 3)

The system according to supplementary 1,
wherein the processor is configured to generate the lifestyle indices and the health state indices by using an image analysis function that performs image recognition processing on image data relating to intake, and an acquisition function that acquires transaction information from an information storage device storing delivery service histories and electronic payment histories, estimate intake items, intake amounts, and nutrition components on the basis of outputs of the image analysis function and the acquisition function, and quantitatively manage the intake contents by using estimation results.

### Application Example 1

### (Supplementary 1)

A system comprising a processor,
wherein the processor is configured to acquire lifestyle data of a user through a terminal device and a portable terminal device including a sensor or a healthcare application, and store acquired biological activity data and location information data as time-series data,
acquire meal image data captured by the user using the terminal device, perform preprocessing on the meal image data by using an image processing program, and convert the preprocessed meal image data into an input format of a machine learning model,
perform inference processing by using an image recognition model including a neural network on the converted meal image data, estimate a meal type, meal components, and nutritional amounts, and structure and record the estimated meal type, meal components, and nutritional amounts as meal content data of the user,
acquire electronic transaction history data of the user via an application programming interface of a transaction service, extract transaction time, transaction amount, transaction type, and transaction location information from the electronic transaction history data, and store the extracted information in a database,
associate the biological activity data, the location information data, the meal content data, and the electronic transaction history data with each other based on time information and location information, and manage the associated data as integrated life log data in a timeline format to perform comprehensive analysis of an exercise amount, meal content, and consumption behavior of the user,
apply evaluation logic to the integrated life log data, calculate a health state index, and generate health state evaluation information including an energy balance, an activity level, and a frequency of high-calorie intake,
generate a prompt sentence including explanatory information described in natural language based on the health state evaluation information and the integrated life log data, and add instructions regarding an output format, a tone, and advice content to the prompt sentence so as to generate a prompt sentence to be input to a generative AI model,
input the prompt sentence to the generative AI model to cause the generative AI model to generate text data, extract concrete advice content and encouragement content corresponding to a health state of the user from the text data, and format the extracted advice content and encouragement content as utterance content of a virtual coach,
generate virtual coach persona information that imitates a preference of the user by reflecting coach profile information including a human image or character attribute selected or set by the user in the prompt sentence, associate the virtual coach persona information with the advice content and the encouragement content, and generate presentation control data, and transmit the presentation control data to the terminal device and present the advice content and the encouragement content from the virtual coach persona to the user by a message display function or a notification function of the terminal device.

### (Supplementary 2)

The system according to supplementary 1,
wherein the processor is configured to
acquire correction input or supplementary input for the meal content data from the terminal device when the user performs the correction input or the supplementary input, update the integrated life log data and the health state evaluation information based on the correction input or the supplementary input, regenerate the prompt sentence based on updated integrated life log data, input the regenerated prompt sentence to the generative AI model, and cause the generative AI model to generate updated advice content and updated encouragement content.

### (Supplementary 3)

The system according to supplementary 1,
wherein the processor is configured to
sequentially execute, at predetermined time intervals based on a scheduler function, processing of acquiring the biological activity data and the electronic transaction history data, processing of analyzing the meal image data, processing of updating the integrated life log data, processing of calculating the health state evaluation information, and processing of generating the prompt sentence to be input to the generative AI model, and continuously provide output data including the advice content and the encouragement content based on the prompt sentence to the terminal device.

### Example 2

### (Supplementary 1)

A system comprising a processor,
wherein the processor is configured to
acquire, via a communication network, user daily-life information input by a user through an information processing program executed on a portable information terminal, and to receive the user daily-life information from the portable information terminal; and
preprocess the received user daily-life information by using a tabular data processing function of an information processing unit so as to convert the user daily-life information into feature information indicating at least one of personality, preference, lifestyle, exercise status, and dietary status of the user; and
convert the feature information into a numerical feature vector by using a machine-learning feature transformation function of an information processing unit, and store the numerical feature vector in association with the user; and
generate a prompt sentence, expressed in natural language, that summarizes at least one of the personality, preference, lifestyle, exercise status, and dietary status of the user, based on the numerical feature vector; and
execute a generative artificial intelligence model by using an information processing unit, with the prompt sentence as an input, so as to generate profile information of a virtual instructor configured to provide advice and encouragement regarding exercise or diet to the user, and to generate one or more advice messages output from the virtual instructor; and
transmit the generated profile information of the virtual instructor and the one or more advice messages to the portable information terminal, and cause the profile information and the one or more advice messages to be presented to the user via a display unit of the portable information terminal; and
acquire evaluation information and feedback information regarding at least one of the virtual instructor and the one or more advice messages from the user via the portable information terminal, and accumulate the evaluation information and the feedback information; and
update at least one of the generative artificial intelligence model and a process of generating the prompt sentence, based on a correspondence among the accumulated evaluation information, the accumulated feedback information, the numerical feature vector, and the prompt sentence, so as to improve generation accuracy of the profile information of the virtual instructor and the one or more advice messages.

### (Supplementary 2)

The system according to supplementary 1,
wherein the processor is configured to
cause the portable information terminal to generate, based on the profile information of the virtual instructor, a visual display object representing a character of the virtual instructor, and
to display the visual display object in combination with the one or more advice messages on a user interface of the portable information terminal.

### (Supplementary 3)

The system according to supplementary 1,
wherein the processor is configured to
dynamically adjust content and output frequency of the one or more advice messages of the virtual instructor, and successively update the prompt sentence to be input to the generative artificial intelligence model, based on at least one of task completion information of the user, behavior history information of the user, and the evaluation information acquired from the user.

### Application Example 2

### (Supplementary 1)

A system comprising a processor,
wherein the processor is configured to
acquire information relating to a user's daily life from measurement apparatuses and information-processing applications, and integrate the acquired information in a time sequence to generate activity indices and nutrition indices, and
analyze voice information and character information of the user by using a natural language processing algorithm and a voice-feature analysis algorithm, estimate an emotional state, and associate the emotional state with the time-sequenced activity indices and nutrition indices and record the association, and
evaluate, based on the activity indices, the nutrition indices, and the emotional state, at least one of goal-achievement status, fatigue level, and preferences of the user, and generate or update an instructor profile indicating personality characteristics, appearance characteristics, and response style of a virtual instructor entity, and
construct a prompt sentence written in a natural language, the prompt sentence describing a current situation of the user, a desired guidance policy, and constraint conditions for a message, on the basis of the activity indices, the nutrition indices, the emotional state, and the instructor profile, and
input the prompt sentence into a generative information generation model, and obtain, from the generative information generation model, response information including at least one of an advice message and an encouragement message to be presented by the virtual instructor entity to the user, and
determine, on the basis of the response information, at least one of a display operation, an audio output, and dialog content of the virtual instructor entity, and present the virtual instructor entity and at least one of the advice message and the encouragement message on a portable information-processing apparatus or a display apparatus, and
adaptively modify, based on feedback information from the user and additional daily-life information acquired after presentation of at least one of the advice message and the encouragement message, contents of a subsequent prompt sentence and the instructor profile to be generated thereafter.

### (Supplementary 2)

The system according to supplementary 1,
wherein the processor is configured to
acquire transaction-related information including at least one of behavior history, purchase history, and location history of the user from a recording medium, integrate meal-candidate information obtained by image analysis with the transaction-related information to estimate intake amounts of nutrients, and incorporate an estimation result into the prompt sentence.

### (Supplementary 3)

The system according to supplementary 1,
wherein the processor is configured to
convert numerical information representing the activity indices, the nutrition indices, and the emotional state into a summary sentence in a natural language in generating the prompt sentence, and generate the prompt sentence stepwise as an instruction sentence optimized for the generative information generation model on the basis of the summary sentence.

What is claimed is:

1. A system comprising:
circuitry configured to:
receive, via a communication interface coupled to a packet-switched network, from a terminal device, time-series sensor data and multimodal input data associated with a user;
apply a first neural network model to the multimodal input data to generate a state classification label representing a detected condition of the user;
generate, based on the time-series sensor data and the state classification label, a numerical feature vector encoding a plurality of attributes of the user;
construct a natural-language prompt sentence incorporating at least a portion of the numerical feature vector and the state classification label;
execute a generative neural network model with the natural-language prompt sentence as an input to produce structured output data comprising an inference result and a persona-adapted output representation; and
transmit, via the communication interface coupled to the packet-switched network, the structured output data to the terminal device for presentation to the user.

2. The system according to claim 1, wherein the circuitry is further configured to: receive, from the terminal device, feedback data indicating an evaluation of the structured output data by the user; and update at least one of the generative neural network model and a process of constructing the natural-language prompt sentence based on a correspondence among the feedback data, the numerical feature vector, and the natural-language prompt sentence.

3. The system according to claim 2, wherein the circuitry is further configured to: aggregate a plurality of feedback data records associated with a plurality of numerical feature vectors; and modify weighting parameters used in constructing the natural-language prompt sentence based on correlation analysis between the plurality of feedback data records and corresponding state classification labels.

4. The system according to claim 3, wherein: the multimodal input data comprises voice data captured by a microphone of the terminal device and text data input by the user; the state classification label represents an emotional state of the user; and the circuitry adjusts content of the structured output data based on the emotional state.

5. The system according to claim 4, wherein the circuitry is further configured to:
apply a sentiment analysis function to the text data to extract a sentiment score; and
concatenate the sentiment score with the emotional state to produce an augmented state classification label used in constructing the natural-language prompt sentence.

6. The system according to claim 1, wherein the circuitry is further configured to:
preprocess the time-series sensor data using a tabular data processing function to generate feature information; and apply a machine-learning feature transformation function to the feature information to produce the numerical feature vector, the machine-learning feature transformation function comprising at least one of encoding categorical variables, scaling numerical values, and transforming text-based fields into numerical representations.

7. The system according to claim 6, wherein the circuitry is further configured to: apply dimensionality reduction to the numerical feature vector to produce a reduced-dimension feature vector; and use the reduced-dimension feature vector in constructing the natural-language prompt sentence.

8. The system according to claim 7, wherein: the time-series sensor data comprises body activity data derived from an accelerometer of the terminal device and location data derived from a positioning receiver of the terminal device; and the feature information comprises aggregated values of body activity and amounts of location movement computed over a defined time window.

9. The system according to claim 8, wherein: the body activity data represents an amount of exercise of the user including at least one of step count, distance traveled, and active minutes; and the circuitry computes lifestyle indices from the aggregated values of body activity and the amounts of location movement.

10. The system according to claim 1, wherein the circuitry is further configured to: receive, from the terminal device, image data captured by a camera of the terminal device; and apply an image analysis neural network to the image data to generate classification labels and estimated quantity values associated with objects depicted in the image data.

11. The system according to claim 10, wherein the circuitry is further configured to: receive transaction data from an external information processing apparatus, the transaction data comprising item identifiers, quantities, and timestamps; and correlate the classification labels with the transaction data using timestamp alignment and string similarity to produce refined estimation values.

12. The system according to claim 11, wherein: the image data represents a meal of the user; the classification labels comprise food category labels; the refined estimation values comprise nutritional content values including calorie estimates and macronutrient ratios; and the circuitry incorporates the nutritional content values into the numerical feature vector.

13. The system according to claim 1, wherein the persona-adapted output representation comprises: profile information defining characteristics of a virtual entity including at least one of a name, a personality description, a communication style, and a coaching style; and one or more guidance messages generated by the generative neural network model in accordance with the profile information.

14. The system according to claim 13, wherein the circuitry is further configured to: construct a second natural-language prompt sentence that instructs the generative neural network model to reformat the inference result in a style defined by the profile information; and execute the generative neural network model with the second natural-language prompt sentence to produce the one or more guidance messages conforming to tone and length constraints specified in the second natural-language prompt sentence.

15. The system according to claim 14, wherein: the profile information is generated based on a user preference setting indicating a preferred person or character; and the virtual entity imitates the preferred person or character in delivering the one or more guidance messages to the user as a coach providing advice and encouragement regarding exercise and diet.

16. The system according to claim 1, wherein the circuitry is further configured to: parse the structured output data to extract labeled sections comprising at least a summary section, a risk identification section, and a recommendation section; and store the labeled sections in an advice log associated with the user and with the underlying time-series sensor data used to generate the structured output data.

17. The system according to claim 16, wherein: the risk identification section identifies health-related concerns based on the numerical feature vector; the recommendation section comprises personalized lifestyle recommendations addressing the health-related concerns; and the circuitry transmits a notification payload to the terminal device indicating availability of updated advice content derived from meal content analysis, exercise tracking, and emotion recognition of the user.

18. A system comprising:
circuitry configured to:
receive, via a communication interface coupled to a packet-switched network, from a terminal device, time-series sensor data and multimodal input data associated with a user;
apply a first neural network model to the multimodal input data to generate a state classification label;
preprocess the time-series sensor data using a tabular data processing function to generate feature information, and apply a machine-learning feature transformation function to the feature information to produce a numerical feature vector;
construct a natural-language prompt sentence incorporating the numerical feature vector and the state classification label, the natural-language prompt sentence comprising explicit instructions specifying a required output format and style constraints;
execute a generative neural network model with the natural-language prompt sentence as an input to produce structured output data comprising an inference result and a persona-adapted output representation including profile information and one or more guidance messages; and
transmit, via the communication interface coupled to the packet-switched network, the structured output data to the terminal device.

19. The system according to claim 18, wherein the circuitry is further configured to: receive feedback data from the terminal device indicating an evaluation of at least one of the persona-adapted output representation and the one or more guidance messages; and perform feedback-aware fine-tuning of the generative neural network model by constructing training examples from the natural-language prompt sentence, the structured output data, and the feedback data, and applying a reward-weighted loss function during gradient-based optimization to adjust model parameters.

20. A method performed by circuitry, the method comprising:
receiving, via a communication interface coupled to a packet-switched network, from a terminal device, time-series sensor data and multimodal input data associated with a user;
applying a first neural network model to the multimodal input data to generate a state classification label representing a detected condition of the user;
generating, based on the time-series sensor data and the state classification label, a numerical feature vector encoding a plurality of attributes of the user;
constructing a natural-language prompt sentence incorporating at least a portion of the numerical feature vector and the state classification label;
executing a generative neural network model with the natural-language prompt sentence as an input to produce structured output data comprising an inference result and a persona-adapted output representation; and
transmitting, via the communication interface coupled to the packet-switched network, the structured output data to the terminal device for presentation to the user.
